(19) United States
(12) Patent Application Publication
(10) Pub. No.: US 2006/0074320 A1
(43) Pub. Date: Apr. 6, 2006

(54) Home ultrasound system

(76) Inventors: Yang Mo Yoo, Seattle, WA (US); Yongmin Kim, Seattle, WA (US); Dong-Gyu Sim, Bellevue, WA (US); Anup Agarwal, Kenmore, WA (US); Fabio Kurt Schneider, Seattle, WA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/213,275

(22) Filed: Aug. 26, 2005

Related U.S. Application Data
(60) Provisional application No. 60/604,883, filed on Aug. 27, 2004.

(51) Int. Cl. A61B 8/14

(52) U.S. Cl. 600/472

(57) ABSTRACT

In embodiments of the present invention, an ultrasound system includes an ultrasound machine, which may be located in a hospital, clinic, vehicle, home, etc., coupled to a remotely located diagnosis station via a communication network. For some embodiments, the ultrasound machine includes an application-specific scan head that has identification information that allows the home ultrasound machine to notify a user whether the attached scan head is appropriate for the type of examination to be performed. For other embodiments, a first stage of beamforming is conducted in reconfigurable hardware and a second stage of beamforming is conducted in programmable software digital signal processor. The diagnosis station may transfer information associated with a scanning protocol for the ultrasound examination to the ultrasound machine via the communication network, and the ultrasound machine may transfer measurement values acquired during the ultrasound examination to the diagnosis station via the communication network.

### BACKGROUND

1. Field

Embodiments of the present invention relate to medical equipment and, in particular, to ultrasound equipment.

2. Discussion of Related Art

There is a growing need for a home-based imaging system that would allow clinicians to have access to patients and be able to make diagnostic decisions remotely. Conventional imaging modalities (e.g., X-rays, computed tomography, magnetic resonance and nuclear medicine) are not portable, and they are more suitable in centralized locations, e.g., hospitals and clinics, due to their size, cost, and training required to operate them. On the other hand, ultrasound imaging is safe, non-invasive, portable, inexpensive, relatively easy to use, and capable of real-time imaging. However, current general purpose ultrasound machines are not appropriate for being used at home because they are still bulky, heavy and expensive. In addition, they need a trained specialist (e.g., sonographer) familiar with their operation and the anatomy of internal organs to scan human body and collect the images and other information for radiologists or other physicians to make diagnostic and therapeutic decisions.

An alternative to the general purpose ultrasound machine is an application-specific ultrasound machine, such as portable ultrasound machines and ultrasonic measuring devices. Many current portable ultrasound machines based on the application-specific integrated circuit (ASIC) technology tend to be relatively small, light, inexpensive, and are mainly used in small hospitals and clinics. Although the size and cost of these portable ultrasound machines have been reduced, they are still difficult to operate and expensive for a home-based imaging system. In addition, several compromises have been made, ranging from the imaging modes supported to the image quality provided. Ultrasonic measuring devices, such as bladder scanners and fetal monitors, for example, significantly reduce the cost and size compared to the portable ultrasound machines as well as general purpose ultrasound machines, but they do not provide real-time ultrasound images that are valuable for remote diagnosis, consultation and/or monitoring/screening.

In addition, since these application-specific ultrasound machines are designed based on fixed-function and hardwired design approaches such as application specific integrated circuit (or ASIC) to reduce the cost and size, they also suffer from limited flexibility, which is one of the key features required for a home-based ultrasound imaging system.

Currently, most medical ultrasound examinations are done in hospitals and clinics using general purpose or portable ultrasound machines by clinicians such as sonographers, radiologists, and physicians, for example, and are interpreted by radiologists or specially-trained physicians. Carrying out a traditional ultrasound scan by an unskilled individual at home is not currently allowed because it may lead to missing pathologies and misdiagnosis.

Another drawback of conventional ultrasound machines concerns beamforming of the received reflected signal. The role of a receive beamformer in a medical ultrasound system is to condition receive signals in order to form high-quality images. In the receive beamformer, a signal received from each individual element in an ultrasound transducer is delayed and then combined together into a single coherent signal.

Digital beamforming has been widely used in diagnostic medical ultrasound systems because it can reduce time delay errors and provide lower side lobes and better image resolution, compared with analog beamforming. In digital beamforming, receive signals are quantized using analog to digital converters (ADCs), delayed using digital circuits and then summed together.

To be able to specify a time delay with accuracy, a high speed ADC is required. From previous studies, a sampling frequency of the ADC, ƒₛ, for accurate digital beamforming is known to be
ƒₛ≧16ƒ₀ (1)
where ƒ₀ is the center frequency of transmitted ultrasound signals. If the center frequency is 5 MHz, the ADC sampling frequency must be higher than 80 MHz, which is still very challenging to support in modern ultrasound machines even with current very large scale integration (VLSI) technology because of the number of ADCs required.

Therefore, in most medical ultrasound machines, receive signals are quantized by ADCs with the sampling frequency of 4ƒ₀ (e.g., 20 MHz) and then interpolated to simulate a sampling frequency of 16ƒ₀. Depending on the interpolation technique used, the digital beamformers can be classified into an interpolator-based beamformer and a phase rotator-based beamformer.

In the interpolator-based beamformer, an interpolator is placed on each channel to interpolate the receive signal. While a coarse time delay is achieved by controlling the first-in first-out (FIFO) memory, a fine time delay is obtained by changing interpolator's coefficients. The interpolator-based beamforming method can achieve an accurate time delay and high contrast resolution. However, it requires a finite impulse response (FIR) filter on each channel for interpolating the receive signal. Furthermore, clock frequency as high as 16ƒ₀ might be needed during interpolation.

Alternatively, the fine time delay is obtained using a phase rotator with the assumption that the receive signal is a narrow-band signal. In the phase rotator-based beamformer, the time delay is converted into a phase value using φ₁=2πƒ₀Δτ₁ where φ₁ and Δτ₁ are the phase value and the time delay for the l^{th} point in receive beamforming, respectively. In this method, the receive signals are quantized similarly as in the interpolator-based beamforming method. Then, complex baseband signals are obtained by demodulating the quantized signals. Alternatively, complex baseband signals can be derived from quantizing the demodulated receive signals. The obtained complex baseband signals are first delayed by the coarse time delay. Then, the phase of the delayed complex baseband signal is rotated by the phase value in order to compensate the phase distortion introduced by performing beamforming on the complex baseband signal.

Since the phase rotator-based beamforming performs beamforming on the complex baseband signal, it does not require interpolation filters and alleviates the high data transfer rate requirement. Thus, the phase rotator-based beamforming would be suitable for a low-cost beamforming technique compared to the interpolator-based beamforming. In addition, further reduction in the hardware complexity can be achieved by dividing the conventional phase-rotator based beamforming into two stages (i.e., coarse time delay adjustment and phase compensation) since they have different hardware requirements (i.e., high data transfer rate and computation, respectively).

Still another drawback of conventional ultrasound systems relates to the signal-to-noise ratio (SNR) and resolution of the system. For example, as described above in medical ultrasound imaging systems, electrical signals are applied to an ultrasonic transducer to generate ultrasound waves, which are then transmitted into the human body for imaging. To obtain high signal to noise ratios (SNR) and good resolution, the electrical signals typically have high peak power and short time duration. Although the time gain compensation (TGC) is applied to the receive signals, it may be difficult to obtain an appropriate SNR for an object deep inside the body due to high attenuation in soft tissues. By increasing the peak power of transmit signals, higher SNRs may be obtained, but it is not desirable because high peak power could potentially damage the ultrasonic transducer and the soft tissues underneath. Therefore, it is necessary to improve the SNRs of medical ultrasound systems without increasing the peak power of transmit signals.

Coded excitation techniques are capable of improving the SNR by increasing the average power of transmit signals instead of the peak power. In coded excitation, an elongated signal, which is encoded with high time-bandwidth (TB) product codes for increasing the average power and preserving the spatial resolution, is transmitted and then the reflected signal from the body is decoded into a short signal by pulse compression. The expected improvement in SNR from coded excitation, GSNR, is given by
GSNR=10 log₁₀ M (2)
where M represents the relative time duration of the elongated transmit signal with respect to that of the conventional short transmit signal. However, it is practically difficult to achieve the above SNR improvement due to the limited transmit power efficiency (TPE) of the encoded transmit signal, which is defined as the ratio of the transmit power available at the output and input of an ultrasonic transducer. Therefore, when selecting an encoding code, the TPE should be considered with other desirable features, such as imaging resolution and transmitter complexity.

Various TB codes, including Chirp, Golay and Barker, have been extensively examined for coded excitation. Among these codes, the Chirp codes can maximize the TPE because they can be designed to have most of their energy within the frequency bandwidth of the ultrasonic transducer. The Chirp codes are commonly weighted by a window function (e.g., Hanning and Chebyshev) to attain acceptable imaging resolution, i.e., narrow mainlobe width (MLW) and low peak sidelobe level (PSL). However, the weighted Chirp codes need a complex transmitter on each channel to amplify their arbitrary values, i.e., a linear power amplifier.

The Golay codes can provide the narrow MLW and minimal PSL with Golay sequences (i.e., +1 and −1). Although the Golay codes do not need complex power amplifiers, paired firings are needed, leading to a reduction in frame rates. In addition, for coherent summation between the complementary pairs, additional hardware is needed to store the results from pulse compression. Moreover, if there is tissue motion during paired firings, severe artifacts are introduced due to the incoherency between the complementary Golay codes.

On the other hand, the Barker codes can provide the narrow MLW and low PSL (e.g., −22 dB with the length of 13) without the need of paired firings and the complex power amplifier. For further reduction in PSL, a sidelobe suppression filter can follow matched filtering when performing pulse compression. However, the Barker codes suffer from the low TPE due to their wide frequency bandwidth that is not matched to that of the ultrasonic transducer. The low TPE results in lower sensitivity and higher temperature in the ultrasonic transducer due to high dissipated power. Therefore, it is desirable to improve the TPE of the Barker codes when using them as an elongated transmit signal in coded excitation.

As described above, the receive signal is decoded by pulse compression to improve the SNR and spatial resolution, particularly in the axial direction. Two types of pulse compression can be applied. In pre-compression, the receive signal is compressed by a pulse compressor on each channel before receive beamforming. This approach can certainly offer the effective compression of the receive signal. However, it requires multiple pulse compressors, resulting in a high complexity in pulse compression. In post-compression, the receive signals from multiple channels are coherently combined together by the receive beamformer, and then the beamformed signal is decoded by a single pulse compressor. Although the post-compression method can reduce the computational complexity in pulse compression significantly, it introduces artifacts in the images due to distortions in the elongated signals caused by dynamic receive focusing during receive beamforming. Therefore, it is desirable to develop an efficient pulse compression method for the Barker codes to achieve effective pulse compression with an acceptable computational complexity.

### SUMMARY OF EMBODIMENTS OF THE INVENTION

A home ultrasound system according to embodiments of the present invention may be capable of adapting to changing clinical needs and/or new applications, assisting non-experts to acquire clinically usable data/images, updating the examination protocols (i.e., scanning, image formation and analysis) from a remote location, supporting new clinical applications and/or adapting to changing clinical needs, supporting an efficient power management for improved portability and longer battery life, and supporting remote diagnosis, consultation and/or monitoring/screening. For some embodiments, the home ultrasound system includes a home ultrasound machine, external computing devices, local storage, central storage, and/or a remotely located diagnosis station. The home ultrasound machine may be used to scan a patient at home and acquire ultrasound data. The acquired ultrasound data may be transferred to the diagnosis station via a communication network. The home ultrasound machine may be located in a clinic, such as a local neighborhood clinic, in a physician's office, and/or in a hospital, such as in a hospital emergency room, for example. The home ultrasound machine also may be located in a vehicle, such as an aid vehicle, for example.

For some embodiments, the home ultrasound machine may include an application-specific scan head, reconfigurable hardware, a programmable processing unit, configuration memory, a power manager, a system controller, a network interface, a user interface, embedded storage, and so on.

The application-specific scan head may be changeable to support different applications. The scan head may include an identification memory that stores information associated with a type for scan head. For some embodiments, the home ultrasound machine may compare the scan head type with a scan head type specified for a particular ultrasound examination and provide an error indication if the scan head type and the scan head type specified for the particular ultrasound examination do not match.

The scan head may transmit an ultrasound signal to a patient and receive a reflected ultrasound signal. The reflected ultrasound signal may be converted to a radio frequency (RF) signal in the scan head. The scan head also may encode the transmit signal with binary phase codes, such as Barker codes, for example. The scan head may use an efficient transmit power (ETP) coding process. The scan head may encode the transmit signal with binary phase codes, such as Barker codes, for example. The ETP-coding process consists of three stages: generation of a principal transmit signal, selection of an encoding kernel, and coding the generated transmit signal with the selected encoding kernel.

For effective pulse compression with a low computational complexity, 2-stage pulse compression is applied to the receive signals. This 2-stage pulse compression consists of a pre-compressor using matched filters and a post-compressor using a single sidelobe suppression filter. In each pre-compressor, a matched filter is used to decode the receive signals coded with the Barker codes to minimize the distortion of the receive signal during receive beamforming. The decoded receive signals are combined together during receive beamforming.

A low-cost digital receive beamformer is provided by dividing a phase-rotator based beamforming into two stages (i.e., pre- and post-beamforming). In the pre-beamforming stage where high data transfer rate is needed, the appropriate complex baseband samples with coarse time delays are selected. The phase compensation requiring high computation capability is performed in the post-beamforming stage. In one embodiment, the pre-beamforming stage is implemented on the low-cost reconfigurable circuit(s) while the post-beamforming stage is implemented on the programmable digital signal processors. Thus, the cost reduction is obtained in the phase-rotator based beamforming by utilizing low-cost reconfigurable circuits and digital signal processors and taking advantage of their hardware reusability.

The reconfigurable hardware may perform pre-beamforming in which a coarse time delay adjustment may be applied to the received signal. The reconfigurable hardware also may perform the first stage of compression in which the received signal may be decoded. The reconfigurable hardware may be programmable gate array (PGA), a field programmable gate array (FPGA), a programmable logic device (PLD), or an application specific integrated circuit (ASIC). The programmable processor may be software on a digital signal processor (DSP).

The programmable processor may perform a second stage of compression in which the peak sidelobe level (PSL) of the coherent beam formed following pre-and post-beamforming is filtered. Then, the beamformed signal is filtered with the sidelobe suppression filter to reduce the PSL of the decoded receive signals. The matched filter used in the pre-compressor can be implemented by using only 2's complement adders because the Barker codes are composed of binary sequences. Thus, the matched filter can be placed in each channel without creating a large computational burden. Only a single sidelobe suppression filter, which can be implemented using complex multipliers as well as adders, is needed in the post-compressor. Therefore, the developed coded excitation technique is a cost-effective solution to improve the SNR in the medical ultrasound systems by enhancing the TPE and minimizing the artifacts from dynamic receive focusing while reducing the necessary hardware complexity.

The programmable processor also may perform back-end processing such as B-mode, spectral Doppler, color-flow and 3D processing, for example. To assist non-clinical people in acquiring clinically usable data and/or images, the programmable processing unit also may be utilized in performing assisted guidance and application-specific analysis by taking advantage of the reusability of the programmable hardware.

The reconfigurable hardware (HW) and the programmable processing unit may be capable of adapting to changing clinical needs and/or new applications by downloading the corresponding configuration information either locally (e.g., flash memory) or via the communication network. This configuration information may be stored in the configuration memory and utilized by the home ultrasound machine.

For longer battery life, the home ultrasound machine may provide an efficient power management based on transducer contract analysis. Furthermore, different levels of power saving modes are supported by adjusting the system parameters as well as by changing the display intensity.

To support remote diagnosis, consultation and monitoring/screening, the beamformed RF data, ultrasound images, and measurement values may be transferred to the diagnosis station by a communication network. The beamformed RF data may be processed by the diagnosis station utilizing signal and image processor(s) or a personal computer (PC) to generate ultrasound images and/or application-specific measurement values in the hospital/clinic.

Alternatively, the beamformed RF data may be processed by external computing devices such as a personal computer (PC), for example, using software and/or hardware at home. The acquired RF data, images and measurement values from the external computing devices may be stored locally and then directly transferred to the hospital/clinic.

In the hospital/clinic, the diagnosis station may be used to review and analyze the transferred ultrasound data and images. Alternatively, the diagnosis station may be used to optimize the parameters for the back-end processing and measurement algorithms to generate better quality images and more accurate measurements. The diagnosis station also may be utilized to generate and transfer the settings/parameters and algorithms for ultrasound data acquisition and/or signal/image processing used in the home ultrasound machine. The ultrasound images and data may be converted to standard formats, such as Digital Imaging and Communications in Medicine (DICOM) format, for example, and transferred into picture archiving and communications system (PACS) for further diagnosis and/or permanent archiving. Using the home ultrasound system according to embodiments of the present invention, ultrasound examinations could be performed at home by non-experts such as patients and their family members, for example, and then acquired ultrasound data including images can be transferred to hospitals for interpretation by radiologists and/or trained clinicians.

### BRIEF DESCRIPTION OF THE DRAWINGS

In the drawings, like reference numbers generally indicate identical, functionally similar, and/or structurally equivalent elements. The drawing in which an element first appears is indicated by the leftmost digit(s) in the reference number, in which:

FIG. 1 is a schematic diagram illustrating a home ultrasound system according to an embodiment of the present invention;

FIG. 2 is a high-level block diagram of the scan head depicted in FIG. 1 according to an embodiment of the present invention;

FIG. 3 is a high-level block diagram of the reconfigurable hardware (HW) depicted in FIG. 1 according to an embodiment of the present invention;

FIG. 4 is a graphical illustration showing an example of how a transmit focusing delay may be calculated in case of strong and weak focusing when transducer elements are configured as a linear array transducer according to an embodiment of the present invention;

FIG. 5 is a graphical illustration showing an example of how focusing time delay for a pre-beamformer and a post-beamformer can be calculated according to an embodiment of the present invention;

FIG. 6 is a high-level block diagram of the programmable processing unit depicted in FIG. 1 according to an embodiment of the present invention;

FIG. 7 a high-level block diagram of the signal and image processor(s) depicted in FIG. 1 according to an embodiment of the present invention;

FIG. 8 is a high-level block diagram of the guidance assister depicted in FIG. 7 according to an embodiment of the present invention;

FIG. 9 a high-level block diagram of the application-specific image analyzer depicted in FIG. 7 according to an embodiment of the present invention;

FIG. 10 a high-level block diagram of the configuration memory depicted in FIG. 1 according to an embodiment of the present invention;

FIG. 11 a high-level block diagram of the network interfaces depicted in FIG. 1 according to an embodiment of the present invention;

FIG. 12 a flowchart illustrating the operation of the power manager depicted in FIG. 1 according to an embodiment of the present invention;

FIG. 13 is a high-level block diagram of the signal and image processor(s) depicted in FIG. 1 according to an embodiment of the present invention;

FIG. 14 a high-level block diagram of the two-stage pulse compression for coded excitation of a transmit signal according to an embodiment of the present invention;

FIG. 15 is a graphical illustration showing Barker codes and their matched filtering according to an embodiment of the present invention;

FIG. 16 is a graphical illustration showing an elongated transmit signal having the Barker codes illustrated in FIG. 15 according to an embodiment of the present invention;

FIG. 17 is a flowchart illustrating efficient transmit power (ETP) coding according to an embodiment of the present invention;

FIG. 18 is a graphical illustration showing an ETP-coded transmit signal according to an embodiment of the present invention;

FIG. 19 is a high-level diagram of a two-stage pulse compression of a transmit signal according to an embodiment of the present invention;

FIG. 20 is a graphical representation of a matched filter output according to an embodiment of the present invention;

FIG. 21 is a graphical representation of a sidelobe suppression filter according to an embodiment of the present invention;

FIG. 22 shows the results from the two-stage pulse compression method for the receive signal according to an embodiment of the present invention;

FIG. 23 is a high-level block diagram illustrating the pre-beamformer processing unit depicted in FIG. 3 according to an alternative embodiment of the present invention

FIG. 24 is a high-level block diagram of the reconfigurable HW depicted in FIG. 1 according to an alternative embodiment of the present invention;

FIG. 25 illustrates an organization of the pre-beamforming delay LUT depicted in FIG. 3 according to an embodiment of the present invention;

FIG. 26 is a high-level block diagram of the post-beamformer processing unit depicted in FIG. 6 according to an alternative embodiment of the present invention;

FIG. 27 illustrates an organization for the post-beamformer LUT depicted in FIG. 6 according to an embodiment of the present invention; and

FIG. 28 illustrates an organization for the post-beamformer LUT depicted in FIG. 6 according to an alternative embodiment of the present invention.

### DETAILED DESCRIPTION OF EMBODIMENTS OF THE INVENTION

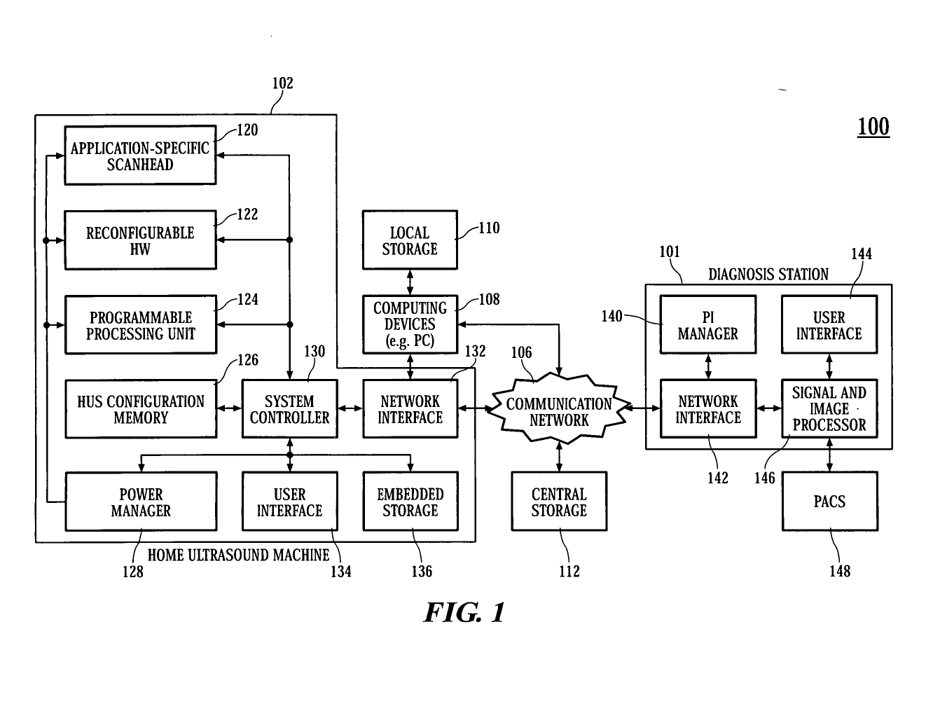

FIG. 1 is a schematic diagram of a home ultrasound system 100 according to an embodiment of the present invention. In the illustrated embodiment, the home ultrasound system 100 includes a home ultrasound machine 102 coupled to a diagnosis station 104 via the communication network 106. In the illustrated embodiment, external computing devices 108 with a local storage 110, and a central storage 112 are also coupled to the home ultrasound machine 102 and the diagnosis station 104.

The illustrated home ultrasound machine 102 includes an application-specific scan head 120, reconfigurable hardware 122, a programmable processing unit 124, home ultrasound system (HUS) configuration memory 126, a power manager 128, a system controller 130, a network interface 132, a user interface 134, and embedded storage 136 operatively coupled to each other. The illustrated diagnosis station 104 includes patient information (PI) manager 140, a network interface 142, a user interface 144 and a signal and image processor 146 operatively coupled to each other. The illustrated diagnosis station also is coupled to a picture archiving and communications system (PACS) 148.

The home ultrasound machine 102 may be used by non-experts (e.g., nurses, patients or their family members) to scan a patient and acquire ultrasound data. The settings and/or parameters for the ultrasound examination may be downloaded from the diagnosis station 104 located in a hospital/clinic. The home ultrasound machine 102 can be adapted to multiple applications by changing the application-specific scan head 120 and/or downloading different configuration information for the reconfigurable HW 122 and the programmable processing unit 124.

For some embodiments, the first stage of beamforming takes place in the reconfigurable HW 122 and the second stage of beamforming takes place in the programmable processing unit 124. Beamforming is used to improve the signal-to-noise-ratio (SNR) and spatial resolution by coherently summing the ultrasound signals from the scan head 120.

The acquired data from the application-specific scan head 120 may be processed in the reconfigurable HW 122 and the programmable processing unit 124, and then the generated ultrasound images and the application-specific measurement values may be transferred to a diagnosis station 104 in the hospital/clinic for remote diagnosis, consultation and/or monitoring/screening. Alternatively, the acquired data after receive beamforming can be directly transferred to the diagnosis station 104 where optimized parameters may be used for better image quality and more accurate measurements.

Alternatively, the acquired data may also be processed in the external computing devices 108, and then the generated ultrasound images and measurement values may be transferred to the diagnosis station 104. In addition, the acquired data including ultrasound images and measurements may be transferred to the central storage 112 for permanent archiving. The acquired data may be converted to standard formats, such as DICOM, for example, and then connected to the PACS 148.

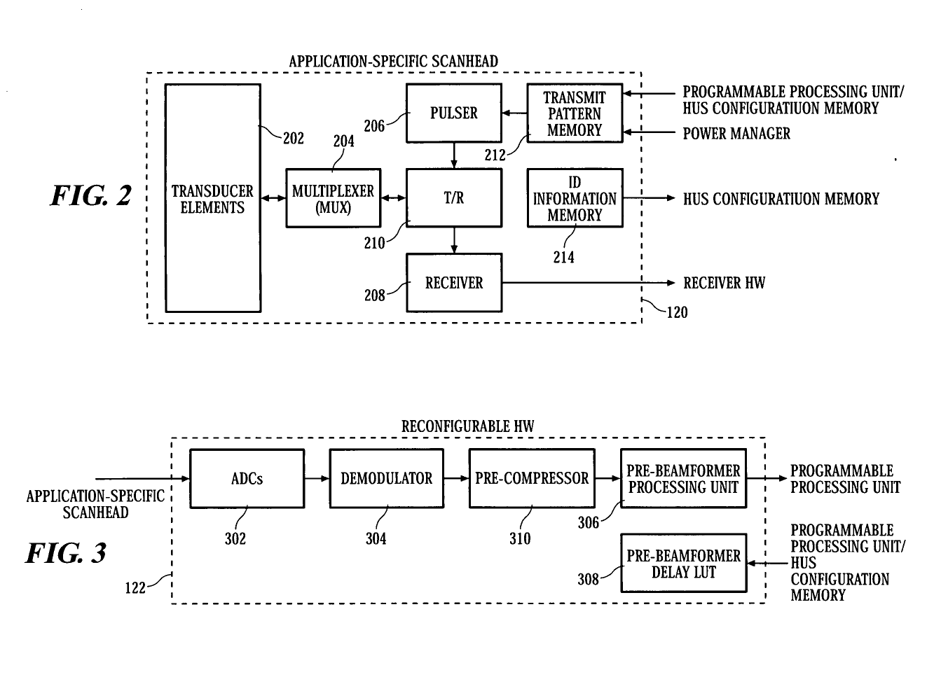

FIG. 2 is a high-level block diagram of the scan head 120 according to an embodiment of the present invention. In the illustrated embodiment, the scan head 120 includes a transducer 202 coupled to an optional multiplexer 204. The multiplexer 204 is coupled to a pulser 206 and a receiver 208 via transmit/receive (T/R) switch 210. A transmit pattern memory 212 is coupled to the pulser 206. The scan head 120 also includes a scan head 120 identification (ID) information memory 214 that would be used to determine if the appropriate transducer 202 has been connected for the specific examination. The information on the transducer to be used for the particular examination would be provided as part of the examination protocol and stored in the HUS configuration memory 126. Thus, the ID information can be compared with the information in the HUS configuration memory 126 to determine if the appropriate transducer has been connected. If incorrect transducer is connected, an error message can be provided to the user via the user interface 134 (e.g., message on display and/or sound).

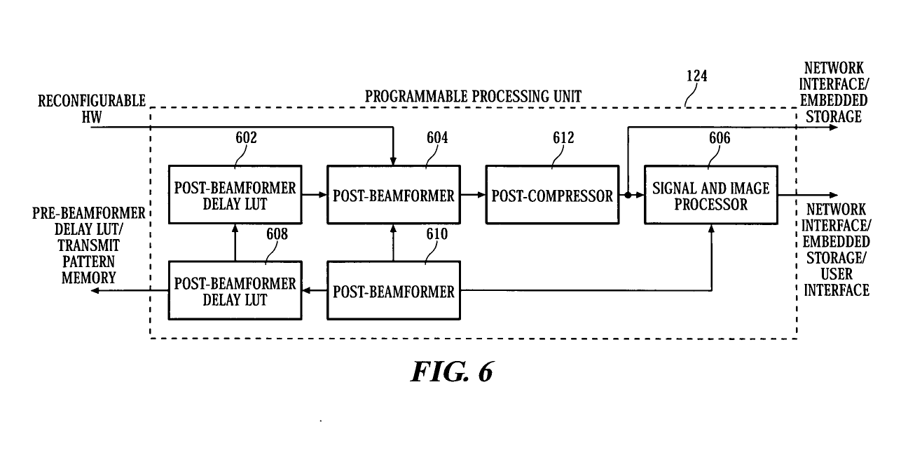

The transmit information (i.e., focusing time delay and transmit power) for the home ultrasound machine 102 may be stored in the transmit pattern memory 212. This information may be initially obtained from the HUS configuration memory 126 and dynamically updated by the beamforming delay calculator (described below with reference to FIG. 6) in the programmable processing unit 124 in real time depending on a specific application and the power mode selected by the user or the remote diagnosis station 104.

The transmit information stored in the transmit pattern memory 212 may be converted to corresponding electrical signals by the pulser 206, and the electrical signals may be used to excite the transducer elements 202. The T/R switch 210 may be used to separate the transmit channel from the receive channel and to protect the receiver 208 circuitry. The multiplexer 204 may be used to multiplex the transducer elements if the total number of transducer elements is different from the number of active transducer elements used for the examination (e.g., linear array and convex array). The ultrasound acoustic signals may be generated by converting the electrical signals into the acoustic waves via transducer elements 202. The reflected acoustic signals are sensed by the transducer elements 202 and converted into radio frequency (RF) electrical signals.

In the receiver 208, the converted RF signals may be amplified in proportion to the depth or the time in order to compensate for signal attenuation (i.e., time gain compensation, TGC) after undergoing low noise amplification (LNA). After LNA and TGC, RF electrical signals may be transferred to the reconfigurable HW 122 for receive beamforming.

FIG. 3 is a high-level block diagram of the reconfigurable HW 122 according to an embodiment of the present invention. In the illustrated embodiment, the reconfigurable HW 122 includes an analog-to-digital converter (ADC) 302 coupled to a demodulator 304. The demodulator 304 is coupled to a pre-beamformer processing unit 306, which is coupled to a pre-beamformer delay lookup table (LUT) 308.

In the reconfigurable HW 122, the RF signals from the application-specific scan head 120 may first be digitized by the ADC 302, e.g., at the sampling frequency of 4ƒ₀ where ƒ₀ is the center frequency of the transducer elements 202. The demodulator 304 may remove the carrier frequency using a demodulation technique, such as quadrature demodulation, for example. In the quadrature demodulation, the quantized RF signal is multiplied with cos(2πƒ₀t) and sin(2πƒ₀t). After low pass filtering, it becomes the baseband signal of complex samples (i.e., I(t)+jQ(t)). Alternatively, the demodulator 304 may remove the carrier frequency by performing a demodulation before digitization.

For some embodiments, in the reconfigurable HW, demodulator 304 is coupled to a pre-compressor 310, which is coupled to a pre-beamformer processing unit 306.

The complex baseband signals then may undergo dynamic receive focusing in order to form high-quality images. In one embodiment, a coarse time delay adjustment may be applied to quantized in-phase and quadrature components of the complex baseband signals.

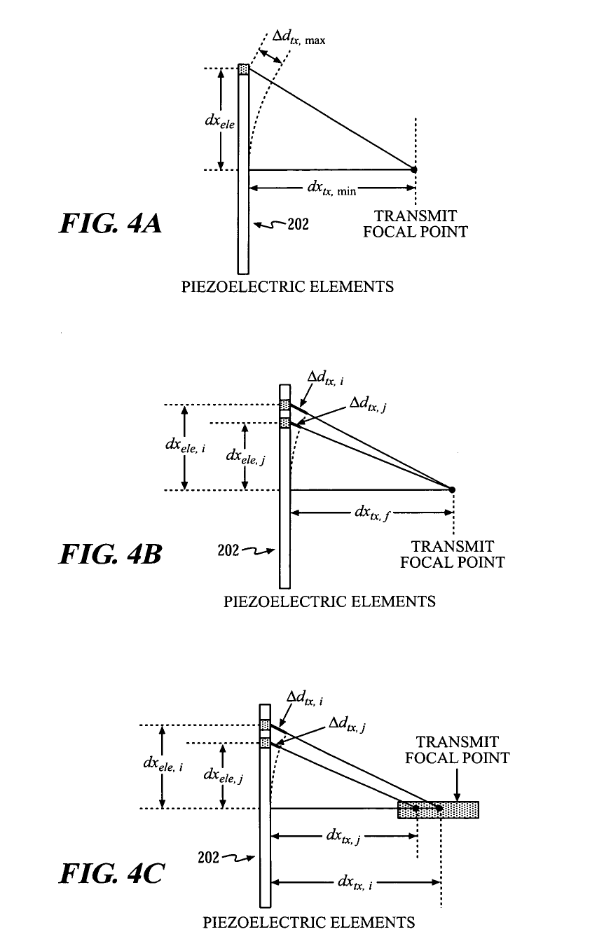

Based on the geometry of transducer elements 202 and the applied beamforming technique, the programmable processing unit 124 may compute transmit focusing time delays. FIG. 4 shows an example illustrating how a transmit focusing delay can be calculated in case of strong and weak focusing when transducer elements 202 are configured as a linear array transducer.

FIG. 4(a) illustrates an estimation of the transmit pattern memory size and calculation of the transmit focusing time delay for (b) strong focusing and (c) weak focusing As shown in FIG. 4(a), the transmit pattern memory 212 size in bits, M, may be determined by the minimal transmit focal point, dz_{tx,min}, and the location of the transducer element that is farthest away from the center, dx_{ele,max}, as follows:

$$\begin{matrix}M = {log}_{2} [ Δ {d}_{tx , max} \times \frac{{f}_{s , tx}}{c} ] \times N \\ = {log}_{2} [ ( \sqrt{d {x}_{ele , max}^{2} + d {z}_{tx , min}^{2}} - d {z}_{tx , min} ) \times \frac{{f}_{s , tx}}{c} ] \times N\end{matrix} \qquad ( 3 )$$

where Δd_{tx,max} is the maximum delay distance, c is the sound velocity, ƒ_{s,tx} is the transmit sampling frequency in the application-specific scan head 120 and N is the number of active transducer elements. In case of strong focusing (e.g., a single focal point at dz_{tx,ƒ}), the time delays for the i^{th} transducer element can be calculated by

$$\begin{matrix}Δ {τ}_{tx , i} = \frac{Δ {d}_{tx , i}}{c} \\ = \frac{\sqrt{d {x}_{ele , i}^{2} + d {z}_{tx , f}^{2}} - d {z}_{tx , f}}{c}\end{matrix} \qquad ( 4 )$$

where Δd_{tx,i} is the delay distance for the i^{th} transducer element. Similarly, the time delay for j^{th} transducer element is given by

$$\begin{matrix}Δ {τ}_{tx , j} = \frac{Δ {d}_{tx , j}}{c} \\ = \frac{\sqrt{d {x}_{ele , j}^{2} + d {z}_{tx , f}^{2}} - d {z}_{tx , f}}{c}\end{matrix} \qquad ( 5 )$$

where Δd_{tx,j} is the delay distance for the j^{th} transducer element.

Strong focusing and corresponding receive beamforming techniques may be appropriate for obtaining better image quality and more accurate measurements. However, their frame rates may be limited because it is difficult to generate more than two scan lines with a single firing. On the other hand, weak focusing and following beamforming techniques can increase the frame rates significantly because they distribute acoustic energy in the transmit focal zone instead of focusing on a single point as shown in FIG. 4(c), so that they can generate multiple scan lines with a single firing (e.g., quad beam and octal beam). In addition, weak focusing can reduce the power consumption due to the reduced number of firings to generate one frame of an ultrasound image. The time delay for the i^{th} transducer element in case of weak focusing is given by

$$\begin{matrix}Δ {τ}_{tx , i} = \frac{Δ {d}_{tx , i}}{c} \\ = \frac{\sqrt{d {x}_{ele , i}^{2} + d {z}_{tx , f}^{2}} - d {z}_{tx , i}}{c}\end{matrix} \qquad ( 6 )$$

where Δd_{tx,i} is the delay distance for the i^{th} transducer element and dz_{tx,i} is the distance between the center of transducer elements and the focal point for the i^{th} transducer element within the transmit focal zone. Similarly, the time delay for j^{th} transducer element is given by

$$\begin{matrix}Δ {τ}_{tx , j} = \frac{Δ {d}_{tx , j}}{c} \\ = \frac{\sqrt{d {x}_{ele , j}^{2} + d {z}_{tx , f}^{2}} - d {z}_{tx , j}}{c}\end{matrix} \qquad ( 7 )$$

where Δd_{tx,j} is the delay distance for the j^{th} transducer element and dz_{tx,j} is the distance between the center of transducer elements 202 and the focal point for the j^{th} transducer element within the transmit focal zone.

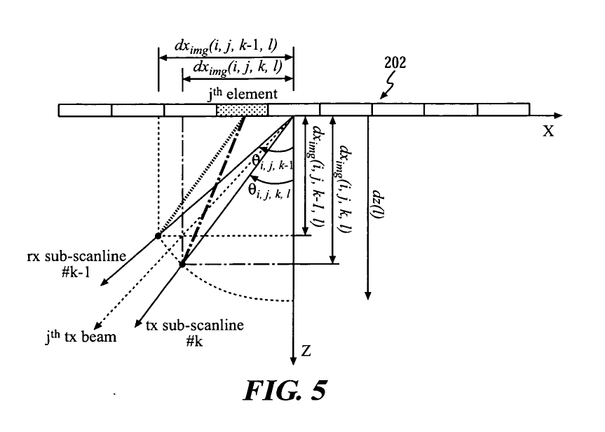

FIG. 5 shows an example illustrating how the receive focusing time delay for the pre-beamformer processing unit 306 (and the post-beamformer described below with reference to FIG. 6) can be calculated in a dual-beam case where two scan lines may be reconstructed with a single firing of the pulser 206 according to an embodiment of the present invention. For calculating the time delay for the i^{th} firing, the j^{th} receive channel, the k^{th} sub-scan line, and the l^{th} axial point, Δτᵣₓ(i, j, k, l), the distance between the axial point or imaging point and the receive element or channel is computed. This distance is given by

$${d}_{rx} ( i , j , k , l ) = \sqrt{{[ d {x}_{img} ( i , j , k , l ) - d {x}_{rx , ele} ( j ) ]}^{2} + {[ d {z}_{img} ( i , j , k , l ) - d {z}_{rx , ele} ( j ) ]}^{2}} \qquad ( 8 )$$

where dx_{img} and dz_{img} are the location of the imaging point in the lateral and axial directions, respectively, and dx_{rx,ele} and dz_{rx,ele} are the location of the receive element. The receive time delay is defined by

$$Δ {τ}_{rx} ( i , j , k , l ) = \frac{{d}_{r x} ( i , j , k , l )}{c} \qquad ( 9 )$$

The time delay for the post-beamformer is obtained by adding the transmit time delay (i.e., Δτₜₓ(i, j, k, l)) and the receive time delay via
Δτ_{tx,rx}(i, j, k, l)=Δτₜₓ(i, j, k, l)+Δτᵣₓ(i, j, k, l) (10)
The pre-beamforming delay represented as the number of samples for the k^{th} sub-scan line, Δ\hat{τ}_{tx,rx}(i, j, k, l), is given by
Δ\hat{τ}_{tx,rx}(i, j, k, l)=T[Δτ_{tx,rx}(i, j, k, l)×ƒ_{s,rx}] (11)
where T[•] is the truncation operator to remove the fractional part and ƒ_{s,rx} is the ADC's sampling frequency. Similarly, the pre-beamforming delay for the k-l^{th} sub-scan line may be obtained for the dual-beam technique.

This time delay represented in Eq. (11) may be computed in real time or may be computed beforehand and stored in memory such as the pre-beamformer delay LUT 308, for example. In one embodiment, the time delays in the pre-beamformer delay LUT 308 may be initially obtained from the HUS configuration memory 126 and dynamically updated in real time by the programmable processor(s) 124. To support various beamforming techniques, multiple pre-beamformer processing units 306 can be integrated into the reconfigurable HW 122 due to the flexibility of the reconfigurable HW 122. The configuration information for the reconfigurable HW 122 may be obtained from the HUS configuration memory 126 when the home ultrasound machine 102 is powered on. This configuration information may be downloaded from the diagnosis station 104 via the communication network 106 or may be updated locally using the external storage device 110 (e.g., flash memory) by the user. The pre-beamformed RF data may be transferred to the programmable processing unit 124 for the fine time delay adjustment and back-end processing.

FIG. 6 is a high-level block diagram of the programmable processing unit 124 according to an embodiment of the present invention. In the illustrated embodiment, the programmable processing unit 124 includes a post-beamformer delay lookup table (LUT) 602 coupled to a post-beam former 604. The post-beamformer 604 is coupled to a signal and image processor 606. A beamforming delay calculator 608 is coupled to the post-beamformer delay LUT 602 and a program memory 610, which is coupled to the signal and image processor 606.

For some embodiments, the post-compressor 612 is performed after post-beamforming and before signal and image processing.

The programmable processing unit 124 may perform three tasks: (1) computation of transmit and receive focusing time delays, (2) post-beamforming in which the phase compensation is applied, (3) post-compression where mismatched filtering is applied, and (4) signal and image processing for generating ultrasound images and performing assisted guidance and application-specific analysis.

For some embodiments, the signal and image processor 606 may generate the ultrasound images from the beamformed RF data.

In some embodiments, the beamforming delay calculator 608 may compute the transmit and receive focusing time delays for the application-specific scan head 120, the reconfigurable HW 122, and the post-beamformer 604 in the programmable processing unit 124. Transmit focusing time delays represented as Eqs. (4) and (5) and two types of the receive focusing time delays (i.e., the pre and post-beamforming delay) represented as Eqs. (10) and (11) may be dynamically computed in the beamforming delay calculator 608 and then transferred to the transmit pattern memory 212, the pre-beamformer delay LUT 308, and the post-beamformer delay LUT 602.

In the post-beamformer 604, the phase of the pre-beamformed complex baseband signals may be adjusted before summation in order to compensate the phase distortion introduced in phase-oration beamforming. After applying the phase compensation by the post-beamformer delay via phase rotation, the complex baseband data from all channels may be coherently combined together in a summation stage. The coherently summed data may be directly transferred to the external computing device 108, the embedded storage 136, and/or the diagnosis station 104 via the communication network 106 for further processing and display. Alternatively, the coherently summed data may be transferred to the signal and image processor 606 for further processing.

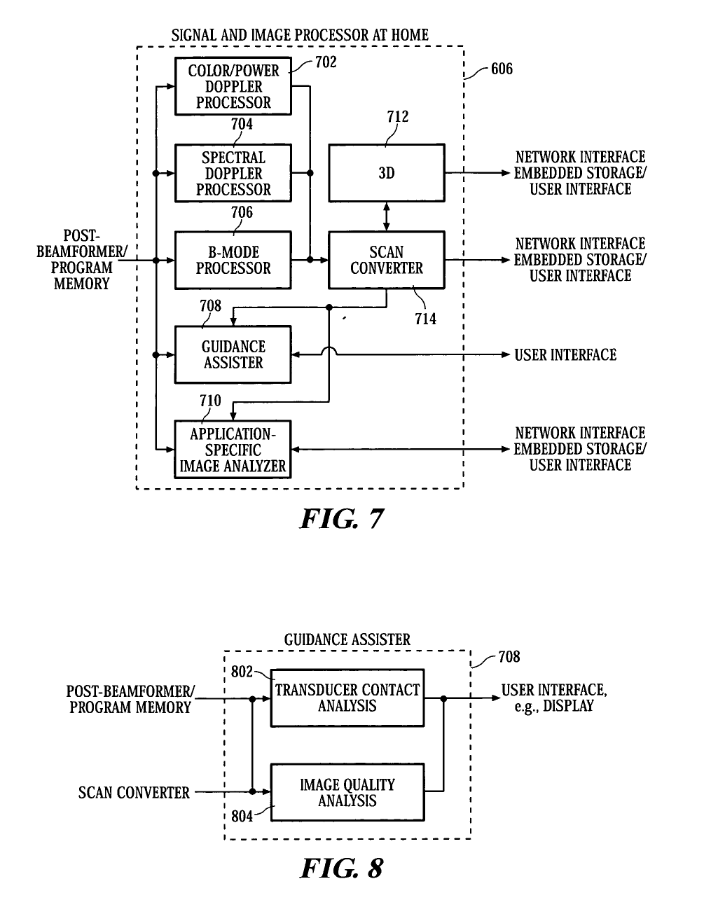

FIG. 7 a high-level block diagram of the signal and image processor(s) 606 according to an embodiment of the present invention. In the illustrated embodiment, the signal and image processor 606 includes a color/power Doppler processor 702, a Doppler processor 704, a B-mode processor 706, a guidance assister 708, an application-specific image analyzer 710, a three-dimensional (3D) processor 712, and a scan converter 714 operatively coupled to each other. From the beamformed RF data, the envelope's magnitude information may be acquired for B-mode, while phase information may be utilized for color, power and spectral Doppler. The Doppler processor 704 may measure whether structures (usually blood) is moving towards or away from the transducer elements 202. The B-mode and color/power Doppler data represented in polar coordinates may be spatially transformed via scan conversion to the geometry and scale of the sector scan on the Cartesian raster output image.

The volumetric data, i.e., 3D data 712, may be reconstructed with the scan-converted B-mode and color/power Doppler data. Alternatively, the 3D data can be obtained directly from the B-mode and color/power Doppler data. The acquired B-mode, spectral,/color/power Doppler and/or 3D data may be stored in the embedded storage 136. Alternatively, the acquired B-mode, spectral/color/power and/or 3D data may be transferred to the central storage 112 and the diagnosis station 104 through the network interface 132. Additionally, the acquired B-mode, spectral/color/power Doppler and/or 3D data may be utilized in the guidance assister 708 and the application-specific image analyzer for less trained operators (e.g., nurses, patients or their family members).

The home ultrasound machine 102 will be typically used by non-experts. It may be challenging for them to acquire appropriate ultrasound images for medical purpose without any guidance. FIG. 8 is a high-level block diagram of the guidance assister 708 according to an embodiment of the present invention that may be used to help non-experts acquire clinically usable ultrasound data. In the illustrated embodiment, the guidance assister 708 includes a transducer contact analysis stage 802 coupled to an image quality analysis stage 804.

In one embodiment, the transducer contact analysis stage 802 may detect scan lines arising from an improper transducer (i.e., transducer elements 202) contact with the underlying tissue of the patient during ultrasound examination. Those scan lines may be identified by measuring the sum of returned energy along each axial direction where the return energy can be estimated by performing inverse time gain compensation (TGC). Alternatively, the transducer contact analysis may be performed with the beamformed RF data without performing inverse TGC.

The evaluation results based on the transducer contact analysis may be indicated to the user via display and/or voice. These contact analysis could be utilized to avoid the excessive exposure of the ultrasound energy to patients. Additionally, these results could also be used to control the transmitting power of the transducer based on the status of the transducer contact. Receive beamforming and image analysis parameters could also be changed when bad contact is detected so that battery life can be extended. For example, when there is a bad transducer contact, the ultrasound machine could be switched to operate in a low-power consumption mode.

At the same time, the image quality may be quantified based on several image quality metrics in real time and the evaluation results may be indicated to the user and/or recorded as part of the image sequence. New guidance stages may be easily added to the guidance assister 708 due to its programmability and flexibility.

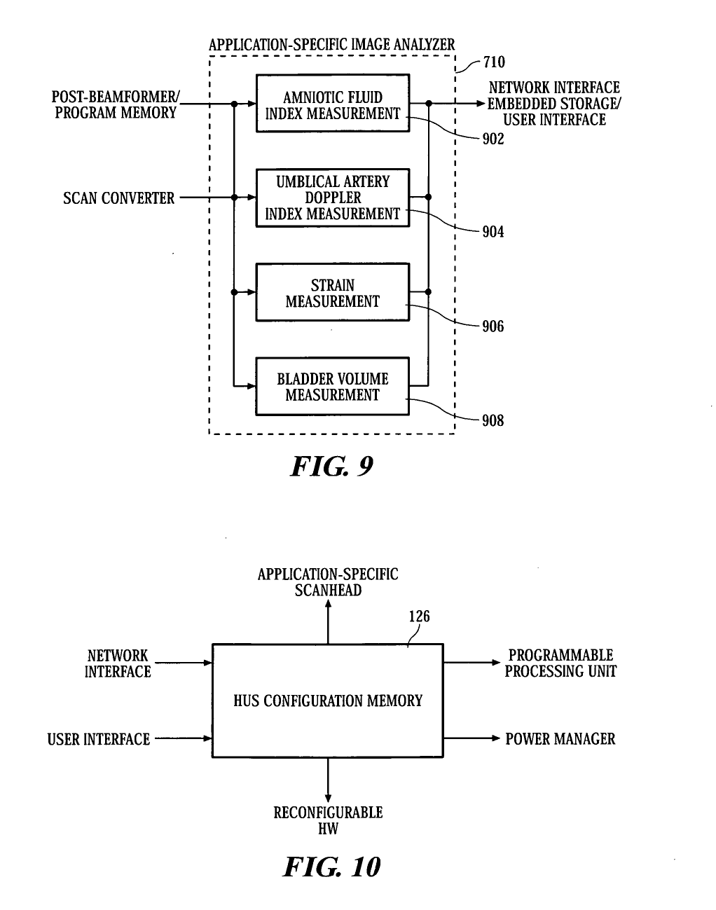

To support multiple clinical applications, application-specific evaluation stages may be integrated into an application-specific image analyzer 710. FIG. 9 is a high-level block diagram of the application-specific image analyzer 710 according to an embodiment of the present invention. In the illustrated embodiment, the application-specific image analyzer 710 includes an amniotic fluid index measurement stage 902, an umbilical artery Doppler index measurement stage 904, a strain measurement stage 906, and a bladder volume measurement stage 908 operatively coupled to each other.

For the illustrated obstetrics and gynecology application, amniotic fluid indexes can be measured, by the amniotic fluid index measurement stage 902, for example, via image segmentation based on the intensity, texture connectivity, and other information. The measurement results may be transferred to the diagnosis station 104 in the hospital/clinic via the network interface 132, and the measurement results may be used for initial diagnosis or screening of patients.

The strain measurement stage 906 may compute straining images based on deformation caused by pressure. Not only the reconstructed strain image but also analysis results, such as locations and sizes of less elastic tissues (potentially cancerous), may be transferred to the diagnosis station 104 in the hospital/clinic via the network interface 132. In addition, the bladder volume measurement stage 908 may be used to estimate the bladder volume by measuring the bladder area. The bladder region may be identified by an image segmentation algorithm and/or other information/techniques. These application-specific analyses may be conducted with ultrasound data as well as several parameters used in TGC, log compression, and/or other stages.

The system configuration parameters for various processing units in the home ultrasound machine 102 may be stored in the HUS configuration memory 126. FIG. 10 a high-level block diagram of the HUS configuration memory 126 according to an embodiment of the present invention. As shown in FIG. 10, these parameters may be downloaded from the diagnostic station 104 via the communication network 106 or may be modified by the user via the user interface 134, which may include external storage devices and/or a keyboard.

For some embodiments, the system configuration parameters may be sent out to the application-specific scan head 120, the reconfigurable HW 122, the programmable processing unit 124, and the power manager 128. The HUS configuration memory 126 may include information such as the initial firing sequence for the pulser 206, initial settings for the pre-beamforming LUT 308 and the post-beamforming LUT 602, configuration information for the reconfigurable HW 122, programs for the programmable processing unit 124, power management information for the power manager 128, and/or application-specific scan head identity information for the ID information memory 214.

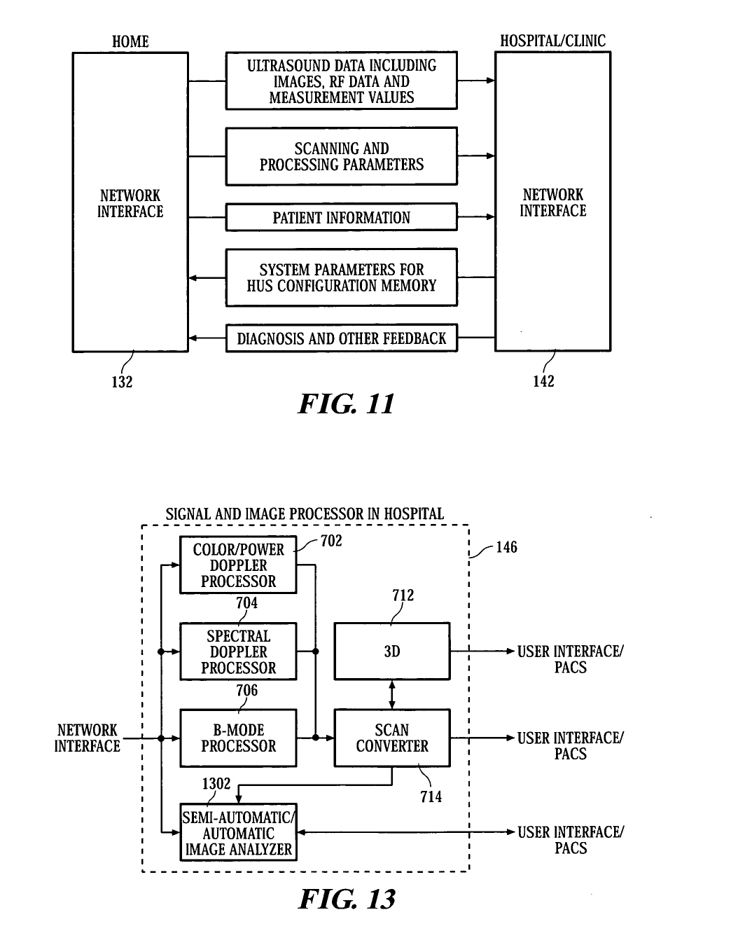

To update examination protocols and support remote diagnosis, consultation and monitoring/screening, the home ultrasound system 102 may provide improved network interfaces. FIG. 11 a high-level block diagram of the network interfaces 132 and 142 according to an embodiment of the present invention. The network interfaces 132 and 142 may facilitate information exchanges between the home ultrasound machine 102 and the diagnosis station 104 located in the hospital/clinic. In addition to various system parameters for HUS configuration memory 126 for the specific examination, diagnosis and other feedback may also be transferred from the diagnosis station 104 located in the hospital/clinic to the home through the network interfaces 132 and 142. The network interfaces 132 and 142 may support the transfer of the following data from home to hospital/clinic patient information, ultrasound data including images, RF data and measurement values, and applied scanning and processing parameters, for example.

Similar to the network interfaces 132 and 142, the user interface 134 may allow information exchanges between the user and the home ultrasound machine 102. In some embodiments, the user interface 134 may be a display for display of the ultrasound images and the current parameters and settings, a keyboard for changing certain parameters or modes in the home ultrasound machine 102, a touch screen for changing certain parameters or modes in the home ultrasound machine 102, sound and/of audio for helping the user to operate the home ultrasound machine 102, for guiding the user's to proper scanning, and for getting user's attention, a communications interface for loading/storing certain parameters in the HUS configuration memory 126 and uploading ultrasound data from the home ultrasound machine 102. Communication interfaces may include standard interfaces (e.g., USB and IEEE 1394).

In some embodiments, the home ultrasound machine provides a power management method based on transducer contact analysis.

In some embodiments the power manager 128 may provide different levels of power modes by changing several system parameters used in the application-specific scan head 120, the reconfigurable HW 122, and the programmable processing unit 124 as well as by adjusting the display intensity. The decision on the power mode to be used may be made by the user based on the trade-off between the desired image quality and the battery time left. Alternatively, this decision can be automatically made by the home ultrasound system 100 based on a predefined setting or by the diagnosis station 104 being operated by the clinician.

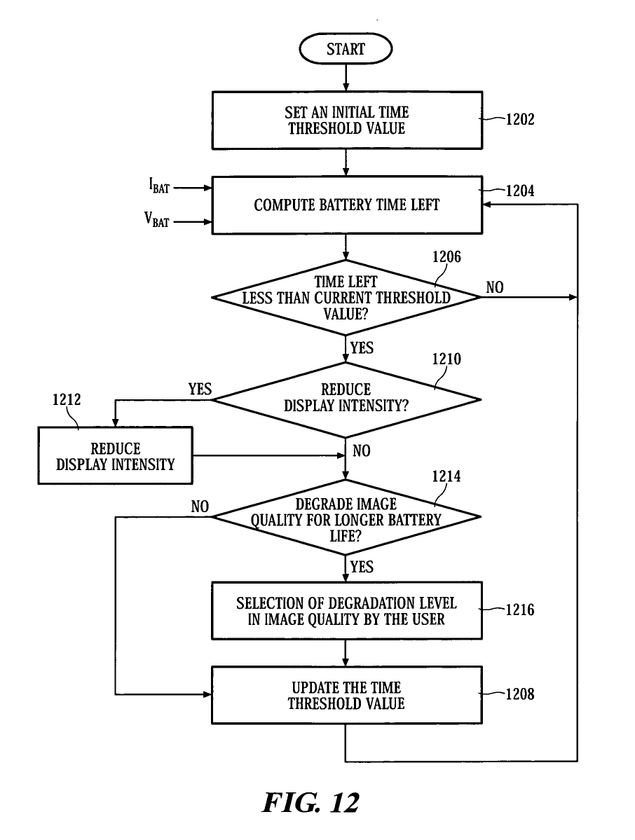

FIG. 12 shows the flowchart on how the power manager 128 works. In a block 1202, an initial time threshold value is first selected.

In a block 1204, the power manager 128 monitors the voltage across the battery source (i.e., V_{BAT}) and the amount of current being provided by the battery source (i.e., I_{BAT}). Using these two parameters, the power being consumed from the battery source is computed, and the energy left in the battery is estimated.

Based on the current power consumption and the battery energy left, the time left in the battery can be estimated. In a block 1206, the process 1200 determines whether the battery time left is more than the predefined threshold value. If the battery time left is not less than the predefined threshold value, the current power mode is not changed and control of the process 1200 passes to a block 1208 in which the time threshold value may be updated.

On the contrary, if the battery time left is less than the current threshold value, the user may be asked to reduce the current power consumption in the home ultrasound machine 102 by reducing the display intensity (block 1210). If the user chooses to reduce the display intensity in a block 1212, the user can reduce the display intensity by a predefined amount and then control passes to a block 1214.

In block 1214, the user may be asked to reduce the current power consumption in the home ultrasound machine 102 by degrading the image quality. If the user chooses to degrade the image quality in block 1216, the user may select a level of image quality degradation in order to prolong the battery life. Different levels of image quality degradation may be achieved by changing the system parameters used in various functional units (e.g., the application-specific scan head 120, the reconfigurable HW 122, and/or the programmable processing unit 124).

The diagnosis station 104 may be used to support remote diagnosis, consultation and/or monitoring/screening. As FIG. 1 illustrates the diagnosis station 104 includes the patient information (PI) manager 140, the network interface 142, the signal and image processor 146 that is similar to the signal and image processor 606 in the home ultrasound machine 102, and the user interface 144 operatively coupled to each other.

For some embodiments, the PI manager 140 may be used to provide the ultrasound examination protocols. In addition, the PI manager 140 may handle the transferred patient information and ultrasound data from the home ultrasound machine 102 to confirm whether the downloaded parameters are appropriately applied during scanning and processing. After this confirmation stage, the transferred ultrasound data including images and measurement values may be transferred to the signal and image processor 146 for further processing and/or diagnosis.

The network interface 142 may handle the communications between the diagnosis station 104 in hospital/clinic and the home ultrasound machine 102.

FIG. 13 is a high-level block diagram of the signal and image processor(s) 146 according to an embodiment of the present invention. The signal and image processor 146 used in the hospital/clinic has similar functionalities as the signal and image processor 606 in the home ultrasound machine 102 except that the signal and image processor 146 includes a semi-automatic/automatic image analyzer 1302 and not the guidance assister 708. While the signal and image processor 146 may be implemented on the programmable processing unit 124 for real-time processing, the signal and image processor 146 in the diagnosis station 104 may be implemented by software and hardware using generic personal computer(s) or programmable processor(s). The signal and image processor 146 also may be used for converting the generated ultrasound image to standard formats (e.g., DICOM) in order to improve the connectivity of the home ultrasound system 100 with the existing PACS 148.

As described above in medical ultrasound imaging systems, electrical signals are applied to an ultrasonic transducer to generate ultrasound waves, which are then transmitted into the human body for imaging. To obtain high signal to noise ratios (SNR) and good resolution, the electrical signals typically have high peak power and short time duration. Although the time gain compensation (TGC) is applied to the receive signals, it may be difficult to obtain an appropriate SNR for an object deep inside the body due to high attenuation in soft tissues. By increasing the peak power of transmit signals, higher SNRs may be obtained, but it is not desirable because high peak power could potentially damage the ultrasonic transducer and the soft tissues underneath. Therefore, it is necessary to improve the SNRs of medical ultrasound systems without increasing the peak power of transmit signals.

Coded excitation techniques are capable of improving the SNR by increasing the average power of transmit signals instead of the peak power. In coded excitation, an elongated signal, which is encoded with high time-bandwidth (TB) product codes for increasing the average power and preserving the spatial resolution, is transmitted and then the reflected signal from the body is decoded into a short signal by pulse compression. The expected improvement in SNR from coded excitation, GSNR, is given by
GSNR=10 log₁₀ M (12)
where M represents the relative time duration of the elongated transmit signal with respect to that of the conventional short transmit signal. However, it is practically difficult to achieve the above SNR improvement due to the limited transmit power efficiency (TPE) of the encoded transmit signal, which is defined as the ratio of the transmit power available at the output and input of an ultrasonic transducer. Therefore, when selecting an encoding code, the TPE should be considered with other desirable features, such as imaging resolution and transmitter complexity.

Various TB codes, including Chirp, Golay and Barker, have been extensively examined for coded excitation. Among these codes, the Chirp codes can maximize the TPE because they can be designed to have most of their energy within the frequency bandwidth of the ultrasonic transducer. The Chirp codes are commonly weighted by a window function (e.g., Hanning and Chebyshev) to attain acceptable imaging resolution, i.e., narrow mainlobe width (MLW) and low peak sidelobe level (PSL). However, the weighted Chirp codes need a complex transmitter on each channel to amplify their arbitrary values, i.e., a linear power amplifier.

The Golay codes can provide the narrow MLW and minimal PSL with Barker sequences (i.e., +1 and −1). Although the Golay codes do not need complex power amplifiers, paired firings are needed, leading to a reduction in frame rates. In addition, for coherent summation between the complementary pairs, additional hardware is needed to store the results from pulse compression. Moreover, if there is tissue motion during paired firings, severe artifacts are introduced due to the incoherency between the complementary Golay codes.

On the other hand, the Barker codes can provide the narrow MLW and low PSL (e.g., −22 dB with the length of 13) without the need of paired firings and the complex power amplifier. For further reduction in PSL, a sidelobe suppression filter can followed matched filtering when performing pulse compression. However, the Barker codes suffer from the low TPE due to their wide frequency bandwidth that is not matched to that of the ultrasonic transducer. The low TPE results in lower sensitivity and higher temperature in the ultrasonic transducer due to high dissipated power. Therefore, it is desirable to improve the TPE of the Barker codes when using them as an elongated transmit signal in coded excitation.

As described above, the receive signal is decoded by pulse compression to improve the SNR and spatial resolution, particularly in the axial direction. Two types of pulse compression can be applied. In pre-compression, the receive signal is compressed by a pulse compressor on each channel before receive beamforming. This approach can certainly offer the effective compression of the receive signal. However, it requires multiple pulse compressors, resulting in a high complexity in pulse compression. In post-compression, the receive signals from multiple channels are coherently combined together by the receive beamformer, and then the beamformed signal is decoded by a single pulse compressor. Although the post-compression method can reduce the computational complexity in pulse compression significantly, it introduces artifacts in the images due to distortions in the elongated signals caused by dynamic receive focusing during receive beamforming. Therefore, it is desirable to develop an efficient pulse compression method for the Barker codes to achieve effective pulse compression with an acceptable computational complexity.

A method and apparatus for a coded excitation technique using efficient transmit power (ETP) coding and 2-stage pulse compression according to embodiments of the present invention improve the SNR and spatial resolution in the home ultrasound system 100. To improve the transmit power efficiency (TPE), an elongated transmit signal based on the binary phase codes (e.g., Barker) may be encoded by the developed ETP coding where the frequency response of transmit signals is matched to that of the ultrasonic transducer.

In some embodiments, the ETP-coding process may include of three stages: generation of a principal transmit signal, selection of an encoding kernel, and coding the generated transmit signal with the selected encoding kernel. For effective pulse compression with a low computational complexity, 2-stage pulse compression may be applied to the receive signals. This 2-stage pulse compression includes of a pre-compressor using matched filters and a post-compressor using a single sidelobe suppression filter.

In each pre-compressor, the matched filter is used to decode the receive signals coded with the binary phase codes (e.g., Barker) to minimize the distortion of the receive signal during receive beamforming. The decoded receive signals may be combined together during receive beamforming. Then, the beamformed signal may be filtered with the sidelobe suppression filter to reduce the peak sidelobe level (PSL) of the decoded receive signals. The matched filter used in the pre-compressor may be implemented by using only 2's complement adders because Barker codes are composed of binary sequences. Thus, the matched filter may be placed in each channel without creating a large computational burden. Only a single sidelobe suppression filter, which can be implemented using complex multipliers as well as adders, may be used in the post-compressor. The developed coded excitation technique is thus a cost-effective solution to improve the SNR in the medical ultrasound systems by enhancing the TPE and minimizing the artifacts from dynamic receive focusing while reducing the necessary hardware complexity.

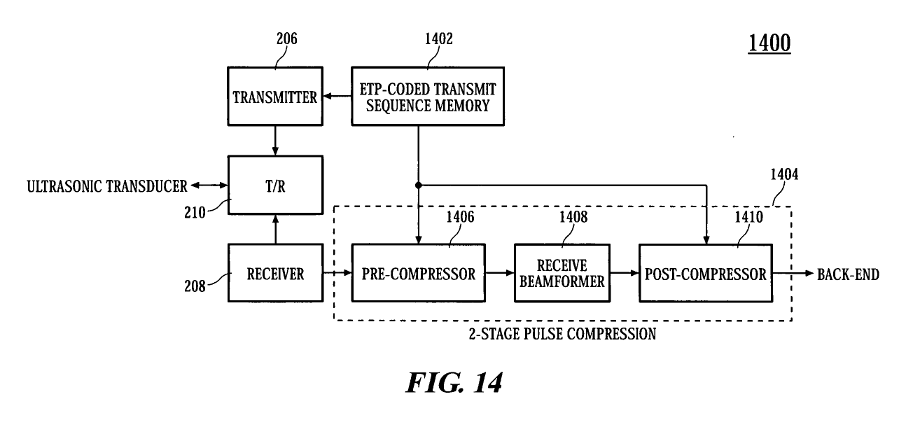

FIG. 14 a high-level block diagram of circuitry 1400 for two-stage pulse compression for coded excitation of a transmit signal according to an embodiment of the present invention. In the illustrated embodiment, the scan head 120 includes an ETP-coded transmit sequence memory 1402 coupled to the transmitter/pulser 206 and to a two-stage pulse compressor 1404. The two-stage pulse compressor 1404 includes a pre-compressor 1406 coupled to a receive beamformer 1408, which is coupled to a post-compressor 1410. The receive beamformer 1408 may include portions of the reconfigurable HW 122 and the programmable processing unit 124.

To improve the TPE, an elongated transmit signal based on the binary phase codes (e.g., Barker) is encoded by the developed ETP coding where the frequency response of transmit signals is matched to that of an ultrasonic transducer. The ETP-coding process consists of three stages: generation of a principal transmit signal, selection of an encoding kernel, and coding the generated transmit signal with the selected encoding kernel. For effective pulse compression with a low computational complexity, two-stage pulse compression is applied to the receive signals.

The pre-compressor 1406 may use matched filters to decode the receive signals coded with the binary phase codes (e.g., Barker) to minimize the distortion of the receive signal during receive beamforming. The decoded receive signals may be combined together in the beamformer 1408 during receive beamforming. The post-compressor 1410 may filter the beamformed signal with the sidelobe suppression filter to reduce the PSL of the decoded receive signals.

For some embodiments, the matched filter used in the pre-compressor 1406 may be implemented by using only two's complement adders because of the property of the binary phase codes. Thus, the matched filter may be placed in each channel without creating a large computational burden. Only a single sidelobe suppression filter, which can be implemented using complex multipliers as well as adders, may be used in the post-compressor 1410. Therefore, the developed coded excitation technique is a cost-effective solution to improve the SNR in the medical ultrasound system 100 by enhancing the TPE and minimizing the artifacts from dynamic receive focusing while reducing the necessary hardware complexity.

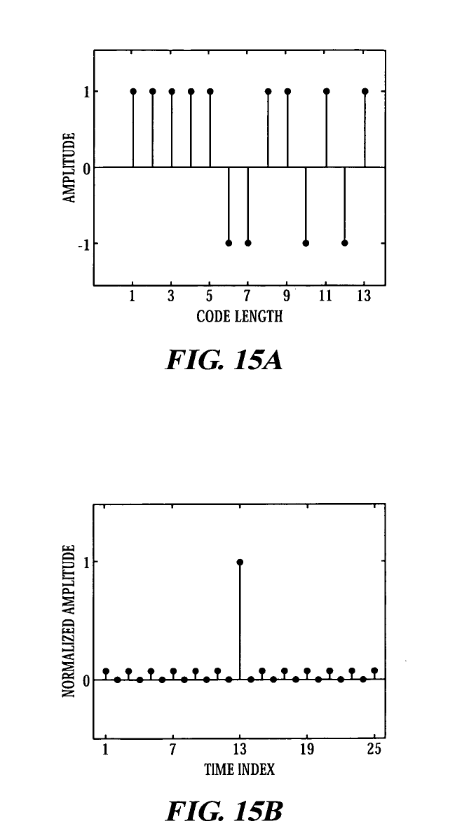

In some embodiments, the ETP-coded transmit signal stored in the ETP-coded transmit sequence memory 1402 and is transmitted through the ultrasonic transducer (i.e., the transducer elements 202). The ETP-coded transmit signal also may be utilized for decoding the receive signal in the two-stage pulse compression. The ETP-coded transmit signal may be generated based on the binary phase codes (e.g., Barker). The PSL of the Barker codes used in the present invention after matched filtering is given by

$$PSL = \frac{1}{M} \qquad ( 13 )$$

where M is the ratio of the time duration of an elongated transmit signal in coded excitation with respect to that of a conventional short transmit signal. FIG. 15 is a graphical illustration showing Barker codes and their matched filtering according to an embodiment of the present invention. FIG. 15(a) illustrates the Barker codes and FIG. 15(b) illustrates their output from matched filtering. As seen in FIG. 15(b), the PSL for the Barker codes with the length of thirteen (i.e., +1, +1, +1, +1, +1, −1, −1, +1, +1, −1, +1, −1, +1) illustrated in FIG. 15(a) is approximately 0.077 (i.e., −22.2 dB).

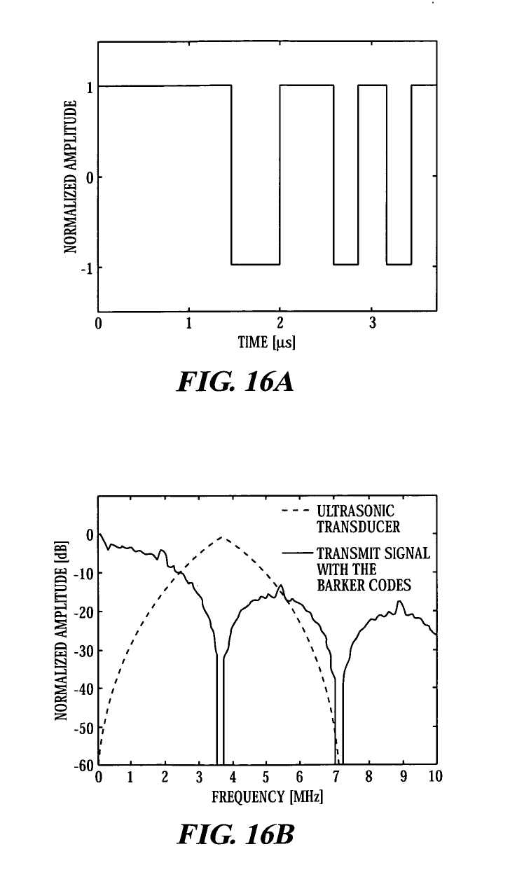

FIG. 16 is a graphical illustration showing an elongated transmit signal having the Barker codes illustrated in FIG. 15 according to an embodiment of the present invention. The elongated transmit signal based on the Barker codes seen in FIG. 15(a) is illustrated in FIG. 16(a) (time domain) and FIG. 16(b) (frequency domain) for a 3.5-MHz ultrasonic transducer. As seen in FIG. 16(b), the TPE of the elongated transmit signal is low so that the transducer sensitivity may be limited and the temperature at the transducer surface may increase due to a large amount of power being dissipated.

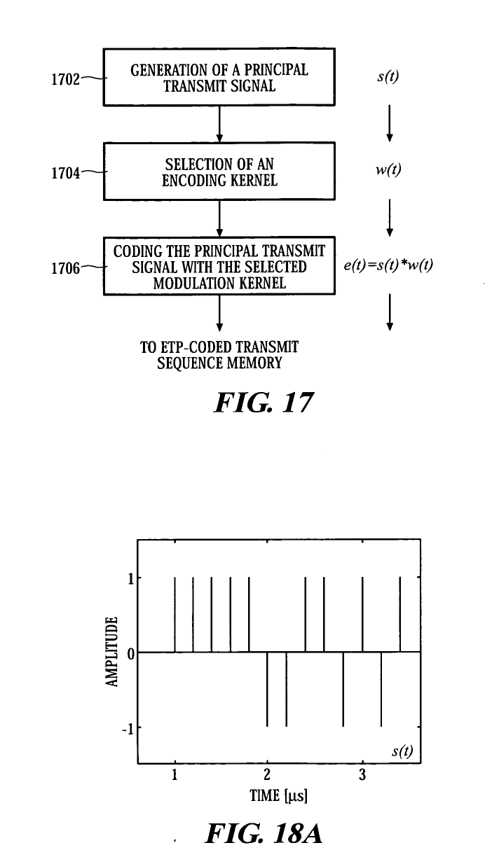

To improve the TPE of the elongated transmit signal based on the Barker codes, we have developed an efficient transmit power (ETP) coding technique. FIG. 17 is a flowchart illustrating efficient transmit power (ETP) coding process 1700 according to an embodiment of the present invention. In a block 1702, a principal transmit signal (i.e., s(t)) based on the Barker codes (i.e., b(k)) is first generated for an ultrasonic transducer with a center frequency of ƒ₀ (i.e.,

$$( i . e . , {f}_{0} = \frac{1}{T} )$$

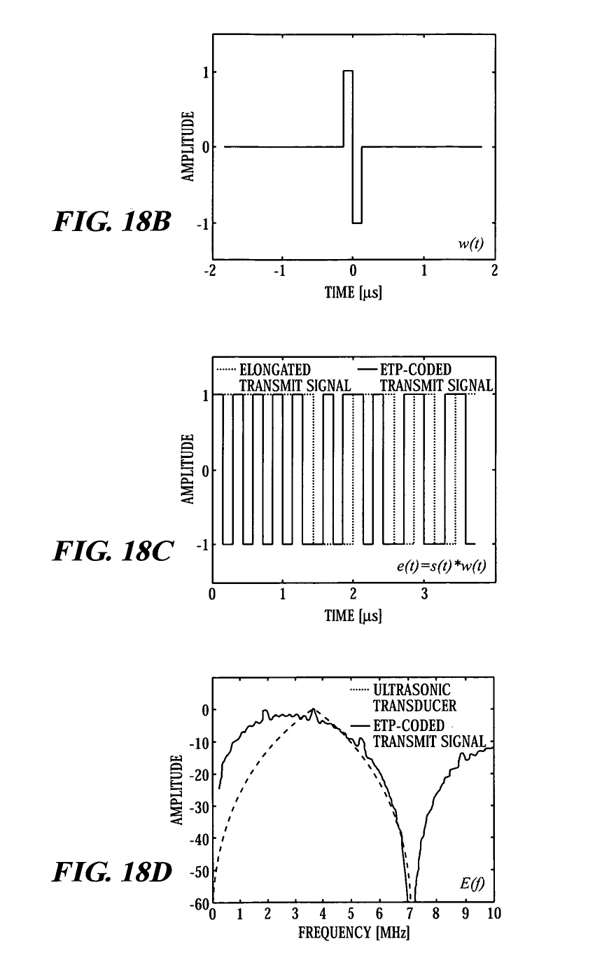

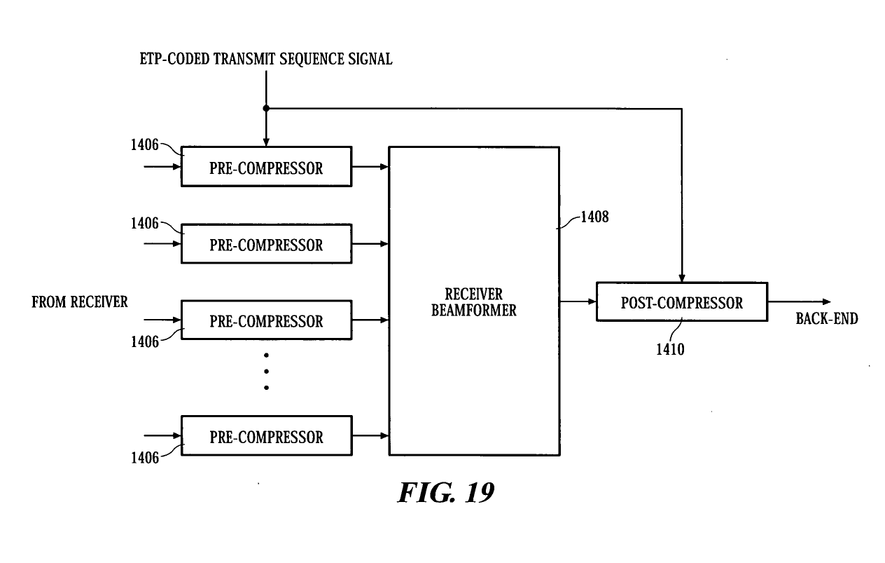

as follows:

$$s ( t ) = {\sum }_{k = 0}^{M - 1} b ( k ) δ ( t - kT ) \qquad ( 14 )$$

where δ(t) is the Dirac delta function. FIG. 18(a) shows an example of principal transmit signals where the Barker codes with the length of thirteen are used for a 3.5-MHz ultrasonic transducer.

In a block 1704, an encoding kernel may be selected. An example encoding kernel for the ETP coding is illustrated in FIG. 18(b) where a bi-phase rectangular window function is shown. This encoding kernel can be any of the following window functions and their combinations: bi-phase Hamming window, bi-phase Hanning window, bi-phase Bartlett window, bi-phase Chebyshev window, bi-phase Kaiser window, and so on. Depending on the desirable ETP improvement and the hardware complexity in the transmitter/pulser 206, a particular encoding kernel may be selected. For example, the bi-phase rectangular window function can be driven by using a simple bipolar pulser, but the expected TPE improvement may be limited due to its high frequency components. On the contrary, the bi-phase Hanning window function may maximize the TPE improvement while it needs a complex linear power amplifier to drive its arbitrary values similar to the Chirp codes.

In a block 1706, the principal transmit signal may be encoded with the selected encoding kernel. FIG. 18(c) illustrates the ETP-coded transmit signal and FIG. 18(d) illustrates the frequency response of the ETP-coded transmit signal with that of the ultrasonic transducer.

For some embodiments, the ETP-coded transmit signal is generated by convolving the principal transmit signal with the selected encoding kernel
e(t)=s(t)*w(t) (15)
where e(t) is the ETP-coded transmit signal, w(t) is the selected encoding kernel and * is the convolution operator. FIG. 18(c) shows an example of the ETP-coded transmit signals in case of e(t) and w(t) being FIG. 18(a) and FIG. 18(b), respectively. The frequency response of the ETP-coded transmit signal is shown in FIG. 18(d) with that of a 3.5-MHz ultrasonic transducer. Compared to the elongated transmit signal seen in FIG. 16(b), the power of the ETP-coded transmit signal may be more concentrated in the frequency bandwidth of the ultrasonic transducer, resulting in higher efficiency. Both ETP-coded and elongated transmit signals may be stored in the ETP-coded transmit sequence memory 1402, and they can be utilized in two-stage pulse compression as described below.

For alternative embodiments, the external computing device 108 may generate the transmit signal, select the encoding kernel, and convolve the transmit signal with the encoding kernel to generate the transmit signal encoded with binary phase codes. The external local storage 110 may then store the transmit signal encoded with the binary phase codes, which the home ultrasound machine 102 may access via the communication network 106 and/or the network interface 132.

FIG. 8 shows the two-stage pulse compression technique according to an embodiment of the present invention where matched filters and a sidelobe suppression filter are used in the pre-compressor 1406 and post-compressor 1410, respectively. The receive signal from the imaging target (i.e., r(t)) may be modeled with the ETP-coded transmit signal as follows
r(t)=α·e(t−Δτ) (16)
where α is the reflection coefficient and Δτ is the time delay corresponding to the location of the target. In the pre-compressor 1406 on each channel, a matched filter is applied to the receive signal based on the corresponding ETP-coded transmit signal as like
r(t)*e(−t)=α·e(t−Δτ)*e(−t) (17)

In case of the bi-phase rectangular window function, the ETP-coded transmit signal may be composed of the binary sequences (i.e., +1 or −1) so that the matched filter can be implemented with two's complement adders. It does not require any complex multipliers. For other window functions, the elongated transmit signal (i.e., without ETP coding) as well as the corresponding ETP-coded transmit signal can be utilized in matched filtering to remove the need for complex multiplers. In fact, the elongated transmit signal may be regarded as a subset of the ETP-coded transmit signal because it can be produced by using the single-phase rectangular window function.

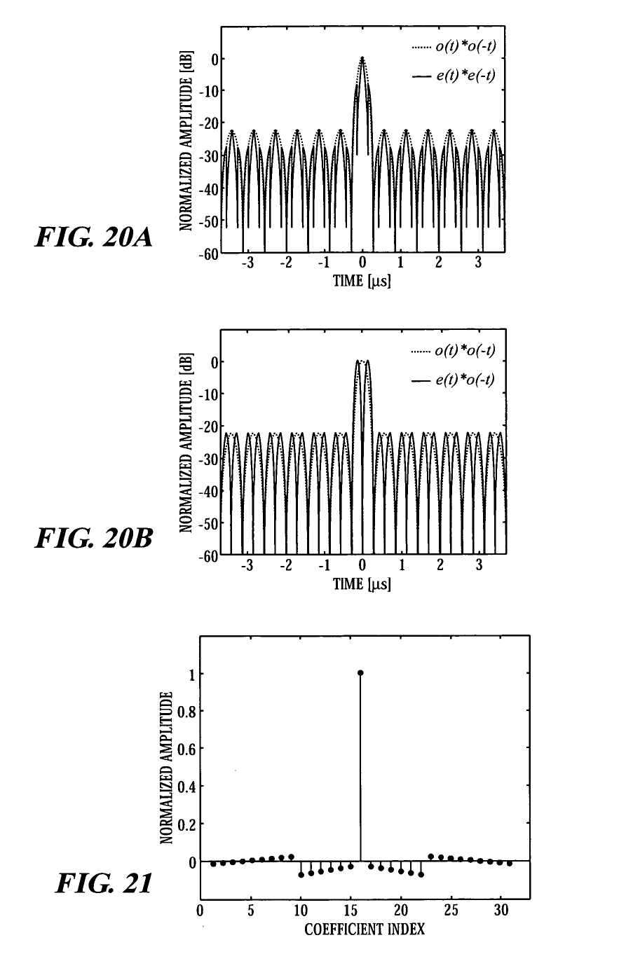

FIG. 20 shows the output from matched filtering when utilizing the ETP-coded and elongated transmit signals as the matched filter kernel. As seen in FIG. 20(a), the ETP-coded transmit signal shows similar results when transmitting the elongated transmit signal after matched filtering, i.e., the PSL is proportional to the length of the transmit signal. Even when the elongated transmit signal (i.e., o(t)) is used as the matched filter kernel, this characteristic may still be preserved as seen in FIG. 20(b). In the illustrated embodiments, o(t) represents the elongated transmit signal.

After matched filtering, the pre-compressed receive signals from all the channels may be coherently combined together in the receive beamformer 1408. For reducing the sidelobes in the lateral direction, apodization can be applied as well. Thus, the beamformed receive signal, d(t), is given by

$$d ( t ) = {\sum }_{n = 0}^{N - 1} b ( n ) \cdot r ( n , t ) * e ( - t ) \qquad ( 18 )$$

where N is the number of channels on the receiver and b(n) is the apodization coefficients.

After receive beamforming, a sidelobe suppression filter may be used in the post-compressor 1410 to remove the sidelobes in the axial direction. The sidelobe suppression filter coefficients can be obtained from the published aricle by Chen et al. entitled “A new algorithm to optimize Barker code sidelobe suppression filters”, IEEE Transactions on Aerospace and Electronic Systems, Vol. 26, pp. 673-677, 1990, by modeling the precompressed receive signal (i.e., C(ƒ)) as the convolution between the mainlobe and sidelobe signals as follows:

$$\begin{matrix}C ( f ) = {C}_{m} ( f ) {C}_{s} ( f ) & ( 19 ) \\ where & \\ {C}_{m} ( f ) = \frac{{sin}^{2} ( π fT )}{{( π fT )}^{2}} & ( 20 ) \\ {C}_{s} ( f ) = M + 1 - \frac{sin ( 2 π fMT )}{2 π fT} & ( 21 )\end{matrix}$$

and Cₘ(ƒ) and Cₛ(ƒ) are the power spectral density of the mainlobe and sidelobe functions, respectively. By performing the inverse Fourier transform of Eq. (21), the sidelobe suppression filter coefficients are obtained. An example sidelobe suppression filter is illustrated in FIG. 21.

Alternatively, the sidelobe suppression filter coefficients may be obtained based on the minimax and genetic optimization approaches as well. The sidelobe suppression filter has arbitary values in its coefficients so that it may use complex multipliers as well as adders. Therefore, it is practically difficult to implement the sidelobe suppression filter with the matched filter in each channel. On the contrary, if both the matched filter and the sidelobe suppression filter are positioned after receive beamforming to reduce the hardware complexity, severe artifacts are introduced due to the distortions in the elongated transmit signals from dynamic receive focusing. However, in the two-stage pulse compression method, a matched filter is implemented in each channel to minimize the signal distortion from dynamic receive focusing while a single sidelobe suppression filter is used to reduce the sidelobes after receive beamforming.

As described above, the sidelobe suppression filter is applied to the decoded receive signal via matched filtering

$$d ( t ) * {c}_{s} ( t ) = [ {\sum }_{n = 0}^{N - 1} b ( n ) \cdot r ( n , t ) * e ( - t ) ] * {c}_{s} ( t ) \qquad ( 22 )$$

where cₛ(t) is the sidelobe suppression filter kernel. If the matched filter has successfully reduced the distortion in the pre-compressed receive signal during receive beamfomring, the convolution operation for sidelobe suppression filtering can be integrated with the summation as follows:

$$\begin{matrix}d ( t ) * {c}_{s} ( t ) = {\sum }_{n = 0}^{N - 1} b ( n ) \cdot r ( n , t ) * e ( - t ) * {c}_{s} ( t ) \\ = {\sum }_{n = 0}^{N - 1} b ( n ) \cdot a \cdot e ( n , t - Δ τ ) * e ( - t ) * {c}_{s} ( t )\end{matrix} \qquad ( 23 )$$

If the sidelobe suppression filter effectively removes the sidelobes, the convolution amongst the receive signals, the matched filter kernel and the sidelobe suppression filter kernel could be given by
e(n,t−Δτ)*e(−t)*cₛ(t)≈δ(n,t−Δτ) (24)

By using Eq. (24), Eq. (23) can be written by

$$d ( t ) * {c}_{s} ( t ) = {\sum }_{n = 0}^{N - 1} b ( n ) \cdot a \cdot δ ( n , t - Δ τ ) \qquad ( 25 )$$

As seen in Eq. (25), after the sidelobe suppression filter, all the information for the imaging target (i.e., the reflection coefficient and distance) can be obtained. FIG. 22 shows the results from the two-stage pulse compression method for the receive signal according to an embodiment of the present invention. FIG. 22(a) illustrates the ETP-coded transmit signal and FIG. 22(b) illustrates the elongated transmit signal as the matched filter kernel. As seen in FIG. 22, the sidelobes have been effectively removed by using the sidelobe suppression filter while the mainlobe broadens slightly.

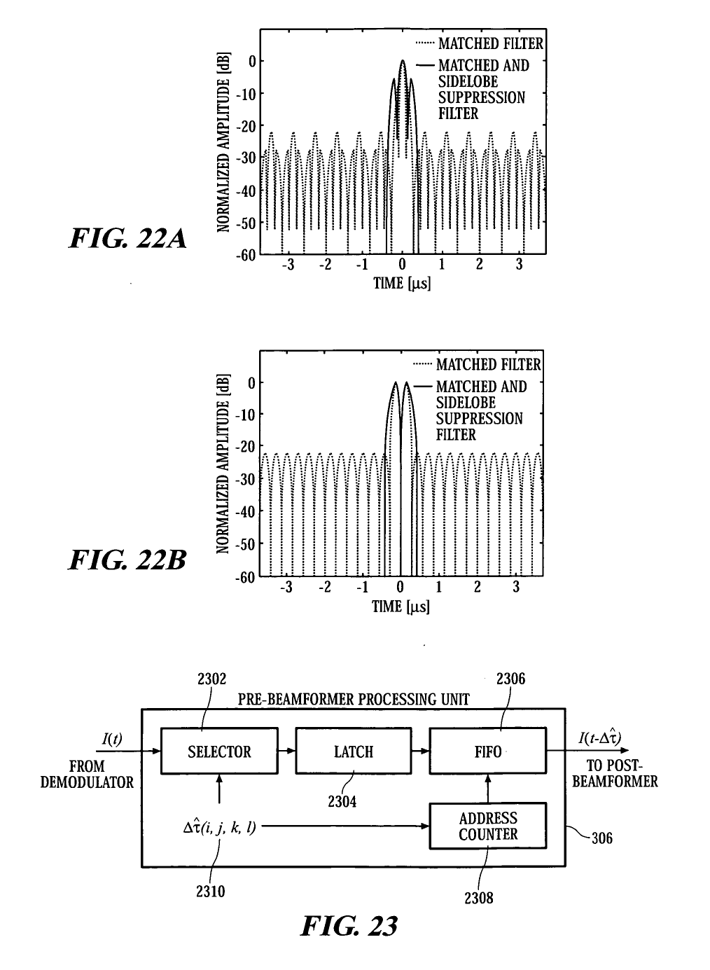

FIG. 23 is a high-level block diagram illustrating the pre-beamformer processing unit 306 according to an alternative embodiment of the present invention. In the illustrated embodiment, the pre-beamformer processing unit 306 includes a selector 2302 coupled to a latch 2304, which is coupled to a first-in-first-out (FIFO) memory 2306. An address counter 2308 is also coupled to the FIFO memory 2306. The pre-beamforming delay, Δ\hat{τ}(i, j, k, l) for the i^{th} firing, the j^{th} receive channel, the k^{th} sub-scanline and the l^{th} imaging point is coupled to the selector 2302 and to the address counter 2308.

The pre-beamforming delay, Δ\hat{τ}(i, j, k, l) for the i^{th} firing, the j^{th} receive channel, the k^{th} sub-scanline and the l^{th} imaging point, is represented as a binary number. In the pre-beamformer processing unit 306, if the delay is ‘0’, the latch 2304 holds the data stored in it. The latch 2304 is updated by the incoming data from the demodulator 304 when the pre-beamforming delay is ‘1’. The latched content is transferred to the first-in first-out (FIFO) memory 2306 for post-beamforming. The pre-beamforming delay may also be utilized for controlling the FIFO memory 2306. In the FIFO memory 2306, only pre-beamformed complex baseband data are sequentially stacked. This allows great coarse delay memory savings and can be implemented in embedded memory of low-cost reconfigurable devices.

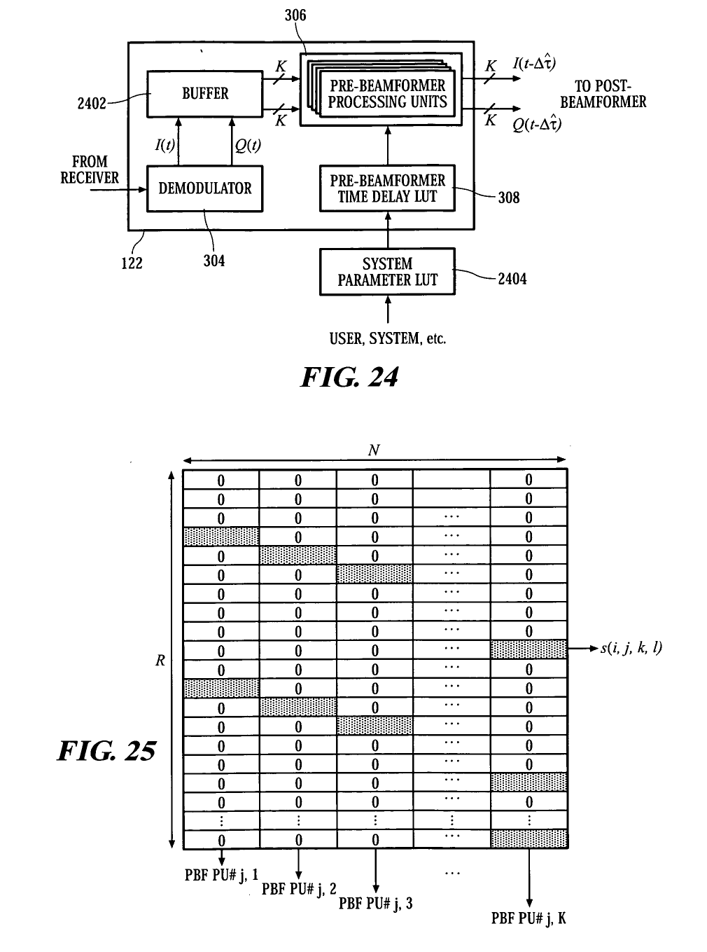

In some embodiments, to support multi-beam and synthetic aperture techniques, multiple pre-beamformer processing units can be integrated into the pre-beamformer. FIG. 24 shows a block diagram for the reconfigurable HW122 with multiple pre-beamformer processing units 306. In the illustrated embodiment, the reconfigurable HW 122 includes the demodulator 304 coupled to a buffer 2402, which is coupled to multiple pre-beamformer processing units 306. The pre-beamformer delay LUT 308 is coupled to the pre-beamformer processing units 306 and a system parameter LUT 2404 is coupled to the pre-beamformer delay LUT 308.

For generating multiple scan lines simultaneously, different pre-beamforming delays are utilized in multiple pre-beamformer processing units 306. As seen in FIG. 24, if there are K pre-beamformer processing units 306, K scan lines can be reconstructed by applying K different time delays. For driving multiple pre-beamformer processing units 306, the buffer 2402 may be needed.

Alternatively, multiple scan lines can be reconstructed by utilizing a single pre-beamformer processing unit with memory in place of the buffer 2402. In order to reconstruct multiple scan lines, the complex baseband data may be stored in the memory and reused in the pre-beamformer processing unit 306. In this embodiment, the multiple scan lines cannot be generated simultaneously. However, only a single pre-beamformer processing unit 306 may be used for supporting multi-beam and synthetic aperture techniques.

FIG. 25 shows an organization of the pre-beamforming delay LUT 308 according to an embodiment of the present invention. The illustrated pre-beamforming delay LUT 308 is organized as R rows and N columns. R represents the total number of axial points corresponding to the penetration depth and R represents a number of receive channels. The total number of axial points corresponding to the penetration depth R can be represented as

$$R = {f}_{s} \times \frac{2 d}{c} \qquad ( 26 )$$

where d is the penetration depth. K represents the number of pre-beamformer processing units 306 or the number of scan lines for multi-beam and synthetic aperture techniques. As can be seen in the pre-beamforming delay LUT 308, only those axial points to be imaged have ‘1’, while the others have ‘0’.

For some embodiments, the size of the pre-beamformer LUT 308 may be reduced by reducing the redundancy in the control words shown in FIG. 25. A control word is a K-bit word for one of the R depths in FIG. 25. A first level of LUT reduction may be obtained by dividing the pre-beamformer LUT 308 into two lookup tables taking advantage that only C out of the 2^{K} possible control words are used. FIG. 29 illustrates the pre-beamformer LUT 308 according to an alternative embodiment of the present invention. In the illustrated embodiment, the pre-beamformer LUT 308 includes a first lookup table (LUT 1) 2902 that may store reduced control words while a second codebook LUT (LUT 2) 2906 may be used to decode the reduced control word to the original K-bit control word. The first lookup table (LUT 1) 2902 may be composed of R reduced control words that are log₂ C-bit long while a second codebook LUT (LUT 2) 2906 is composed of log₂ C K-bit long codes.

For some embodiments, the combination of variable length coding (VLC) and run length coding (RLC) can be used to further reduce the size of the LUT 1 2902. When VLC/RLC encoding is used, the first lookup table (LUT 1) 2902 is coupled to a RLC/VLC decoder 2904, which may provide RLC/VLC decoding of the control words. The RLC/VLC decoder 2904 is coupled to the second lookup table (LUT 2) 2906, which may store a code book for the decoded the reduced control words.

Depending on the home ultrasound system 100 specification and complexity, the number of imaging points in the axial direction can vary. For a high-end home ultrasound system 100 or a high quality image, a large number of imaging points (e.g., 4096 points) can be used. A small number of imaging points (e.g., 512 points) can be used for a cost-effective portable home ultrasound system 100 in order to reduce the size of the FIFO memory 2306 in the pre-beamformer processing unit 306 and the computational complexity in the post-beamformer 604.

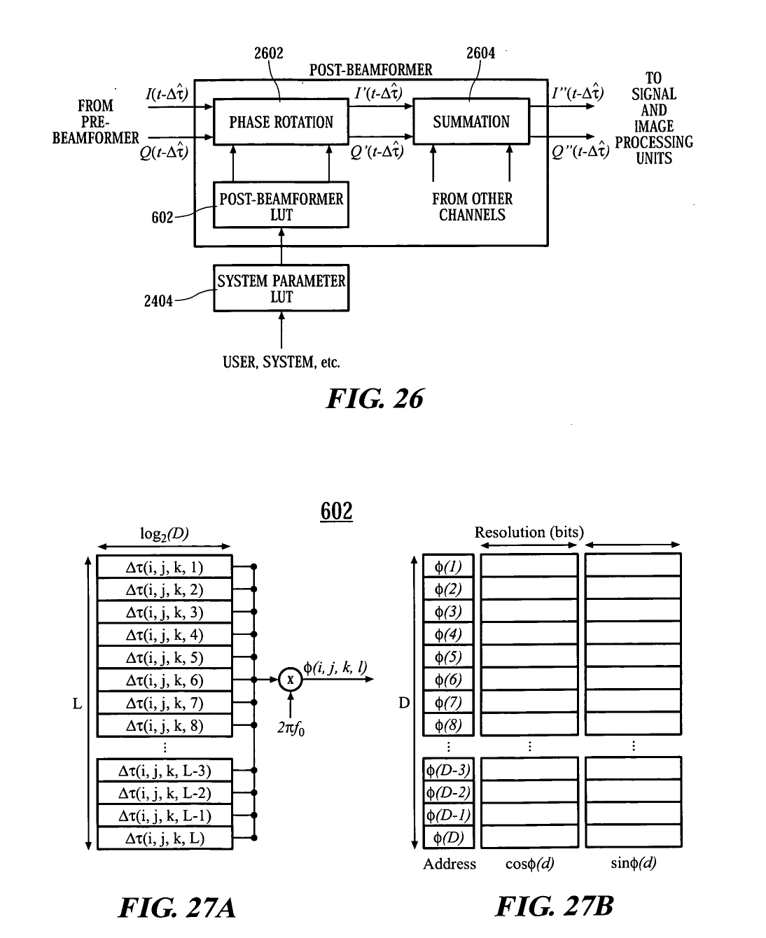

FIG. 26 is a high-level block diagram of the post-beamformer processing unit 604 according to an alternative embodiment of the present invention. In the illustrated embodiment, the post-beamformer processing unit 604 includes a phase rotation stage 2602 coupled to a summation stage 2604. The post-beamforming LUT 602 is coupled to the phase rotation stage 2602 and the system parameter LUT 2404 is coupled to the post-beamforming LUT 602.

For some embodiments, the phase rotation stage 2602 receives the complex baseband signals from the reconfigurable HW 122 (or pre-beamformer) and adjusts the phase of the pre-beamformed complex baseband signal. In one embodiment, the phase rotation can be represented by

$$\begin{matrix}[ \begin{matrix}{I}^{′} ( t - Δ τ ) \\ {Q}^{′} ( t - Δ τ )\end{matrix} ] = \\ [ \begin{matrix}cos ( 2 π {f}_{0} Δ {τ}_{POS\_i , j , k , l} ) & sin ( 2 π {f}_{0} Δ {τ}_{POS\_i , j , k , l} ) \\ - sin ( 2 π {f}_{0} Δ {τ}_{POS\_i , j , k , l} ) & cos ( 2 π {f}_{0} Δ {τ}_{POS\_i , j , k , l} )\end{matrix} ] \cdot [ \begin{matrix}I ( t - Δ {\hat{τ}}_{i , j , k , l} ) \\ Q ( t - Δ {\hat{τ}}_{i , j , k , l} )\end{matrix} ]\end{matrix} \qquad ( 27 )$$

where Δτ_{POS}_{_{—}}_{i,j,k,l} is the post-beamformer delay for the i^{th} firing, the j^{th} receive channel, the k^{th} sub-scanline and the l^{th} axial point. The post-beamformer delay is given by
Δτ_{POS}(i, j, k, l)=Δτ_{POS}_{_{—}}_{i,j,k,l}=Δτᵣₓ(i, j, k, l)−Δτₜₓ(i, j, k, l) (28)

The phase rotation can be facilitated using complex multiplication instructions available in the programmable processing unit 124. One or more post-beamformer phase delays and/or phase compensation values may be computed in real time using a Coordinate Rotation Digital Computer (CORDIC) algorithm. Alternatively, as described earlier, the post-beamformer phase compensation values may be computed in advance, stored in the post-beamformer LUT 602, and utilized later in computing the phase rotation.

The post-beamformer LUT 602 may be organized in several ways. FIG. 27 illustrates an organization for the post-beamformer LUT 602 according to an embodiment of the present invention. In the illustrated embodiment, the post-beamformer LUT 602 includes two levels: the post-beamforming delay LUT, which is shown in FIG. 27(a), and the generic cosine and sine LUT, which is shown in FIG. 27(b). The post-beamforming delay corresponding to required phase compensation may be stored in the first post-beamforming delay LUT shown in FIG. 27(a) and then utilized for selecting the corresponding cosine and sine values in the second post-beamforming delay LUT, which is shown in FIG. 27(b).

The illustrated post-beamformer LUT 602 includes L rows corresponding to the number of imaging points and log₂ D columns whereas D represents the precision of the computed cosine and sine values in the second post-beamforming delay LUT shown in FIG. 27(b). The post-beamformer delay may first be converted into the corresponding phase value, and this value may be used for referring to the cosine and sine values in the second post-beamforming delay LUT shown in FIG. 27(b). The cosine and sine values may be computed for the equally-separated phases from 0 to 2π radians.

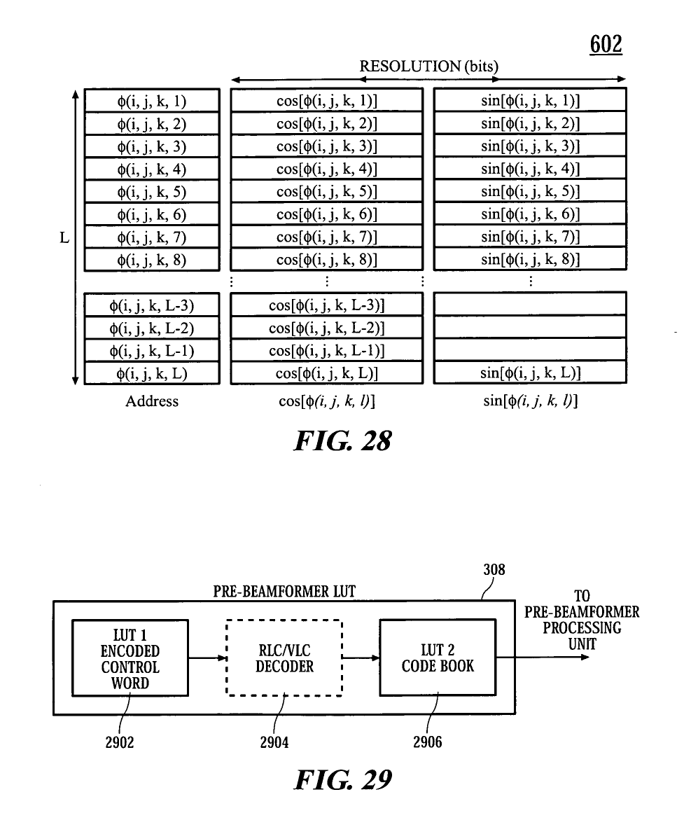

FIG. 28 illustrates an organization for the post-beamformer LUT 602 according to an alternative embodiment of the present invention. In the embodiment illustrated in FIG. 28, the post-beamformer delay LUT 602 is organized with a single level LUT in which actual cosine and sine values corresponding to the phase values for the post-beamforming delay are sequentially stored.

After the phase of the complex baseband signal is adjusted, as specified by the post-beamformer LUT 308, for example, the complex baseband data from all channels may be coherently combined together in the summation stage 2604.

Although some embodiments have been described with reference to an ultrasound machine being located in a home, embodiments are not so limited. Fore example, the ultrasound machine may be located in a clinic, such as a local neighborhood clinic, in a physician's office, and/or in a hospital, such as in a hospital emergency room, for example. The ultrasound machine also may be located in a vehicle, such as an aid vehicle, for example

In the above description, numerous specific details, such as, for example, particular processes, materials, devices, and so forth, are presented to provide a thorough understanding of embodiments of the invention. One skilled in the relevant art will recognize, however, that the embodiments of the present invention may be practiced without one or more of the specific details, or with other methods, components, etc. In other instances, structures or operations are not shown or described in detail to avoid obscuring the understanding of this description.

Reference throughout this specification to “one embodiment” or “an embodiment” means that a particular feature, structure, process, block, or characteristic described in connection with an embodiment is included in at least one embodiment of the present invention. Thus, the appearance of the phrases “in one embodiment” or “in an embodiment” in various places throughout this specification does not necessarily mean that the phrases all refer to the same embodiment. The particular features, structures, or characteristics may be combined in any suitable manner in one or more embodiments.

The terms used in the following claims should not be construed to limit embodiments of the invention to the specific embodiments disclosed in the specification and the claims. Rather, the scope of embodiments of the invention is to be determined entirely by the following claims, which are to be construed in accordance with established doctrines of claim interpretation.

1. An apparatus for performing ultrasound examination of a patient, the apparatus to divide conventional phase-rotator based beamforming into two stages, the apparatus comprising:
reconfigurable logic to perform a first stage of beamforming on a reflected ultrasound signal, the reflected ultrasound signal being reflected off the patient, the reflected ultrasound signal having multiple channels associated with multiple active transducer elements; and
a programmable processor to perform a second stage of beamforming on the reflected ultrasound signal.

2. The apparatus of claim 1, wherein the reconfigurable logic comprises a programmable gate array (PGA), a field programmable gate array (FPGA), a programmable logic device (PLD), and/or an application specific integrated circuit (ASIC).

3. The apparatus of claim 1, wherein the programmable processor comprises a digital signal processor.

4. The apparatus of claim 3, wherein the programmable processor comprises software on a digital signal processor.

5. The apparatus of claim 1, wherein the reconfigurable logic comprises:
circuitry to digitize the time gain compensated RF signal; and
circuitry to demodulate the digitized RF signal and to produce for each channel a complex baseband signal from the demodulated signal, wherein each complex baseband signal includes an in-phase component and a quadrature component.

6. The apparatus of claim 5, wherein the reconfigurable logic comprises a lookup table having stored therein information associated with a time delay for the baseband signals.

7. The apparatus of claim 5, wherein the reconfigurable logic comprises circuitry to calculate a time delay for the baseband signals.

8. The apparatus of claim 6 or 7, wherein the reconfigurable logic comprises circuitry to apply a time delay adjustment to the complex baseband signals based on the calculated time delay.

9. The apparatus of claim 8, wherein the circuitry to apply the time delay adjustment to the complex baseband signals comprises:
a latch;
a first-in-first-out (FIFO) buffer; and
an address counter,
wherein the latch is to hold the complex baseband signals from the demodulator if the delay is a logical “zero” and to transfer the complex baseband signals from the demodulator to the first-in-first-out (FIFO) buffer if the time delay is a logical “one,” and wherein the address counter is to use the time delay to sequentially stack the complex baseband signals in the first-in-first-out (FIFO) buffer.

10. The apparatus of claim 8, wherein the programmable processor is to calculate phase compensation values for the time delay adjusted complex baseband signals.

11. The apparatus of claim 8, wherein the programmable processor is to calculate a time delay for the baseband signals.

12. The apparatus of claim 8, wherein the programmable processor comprises a lookup table having stored therein information associated with phase compensation values for the time delay adjusted complex baseband signals.

13. The apparatus of claim 10, 11, or 12, wherein the programmable processor is to adjust a phase of the time delay adjusted complex baseband signals based on the phase compensation values.

14. The apparatus of claim 13, wherein the programmable processor is to sum the time delayed and phase compensated baseband signal into a coherent beam.

15. An apparatus for performing ultrasound examination of a patient, comprising:
a scan head to transmit a transmit signal being encoded using binary phase codes and to receive a reflected ultrasound signal from the patient;
reconfigurable logic having a first compression stage on each channel; and
a programmable processor to perform a second compression stage,
wherein the first compression stage is to decode the encoded reflected ultrasound signal,
wherein the reconfigurable logic and programmable processor are to form the decoded reflected ultrasound signal into a coherent beam, and
wherein the second stage of compression is to filter a peak sidelobe level of the coherent beam.

16. The apparatus of claim 15, wherein the reconfigurable logic comprises a matched filter to decode the encoded reflected ultrasound signal.

17. The apparatus of claim 16 wherein the matched filter comprises two's complement adders.

18. The apparatus of claim 15, wherein the binary phase codes comprise Barker codes.

19. The apparatus of claim 15, wherein the programmable processor further comprises data stored therein to, when accessed by a machine, cause a sidelobe suppression filter to be applied to the coherent beam.

20. The apparatus of claim 15, wherein the programmable processor further comprises data stored therein to cause the coherent beam to undergo apodization.

21. The apparatus of claim 15, further comprising:
an external computing device to:
generate a transmit signal;
select an encoding kernel; and
convolve the transmit signal with the encoding kernel to generate a transmit signal encoded with binary phase codes; and
external memory to store the transmit signal encoded with the binary phase codes.

22. The apparatus of claim 15, wherein the programmable processor further comprises data stored therein to cause the home ultrasound machine to:
generate a transmit signal;
select an encoding kernel; and
convolve the transmit signal with the encoding kernel.

23. The apparatus of claim 22, wherein the encoding kernel comprises a window selected from a bi-phase rectangular window, a bi-phase Hamming window, a bi-phase Hanning window, a bi-phase Bartlett window, a bi-phase Chebyshev window, and a bi-phase Kaiser window.

24. An apparatus for performing ultrasound examination of a patient, comprising:
a scan head to receive a reflected ultrasound signal from the patient, wherein the scan head includes an identification memory having stored therein information associated with a type for the scan head;
a configuration memory having stored therein information associated with a type for a scan head for a predetermined ultrasound examination and
a controller to compare the information associated with the scan head type stored in the identification memory with the information associated with the scan head type stored in the configuration memory and to provide an error indication if the information associated with the scan head type stored in the identification memory does not match the information associated with the scan head type stored in the configuration memory.

25. The apparatus of claim 24, wherein the scan head further comprises:
a transmitter; and
a transmitter memory having stored therein information associated with a firing sequence and/or transmit power for the transmitter, wherein the transmitter is to generate a radio frequency (RF) signal using the information associated with a firing sequence and/or transmit power stored in the transmitter memory.

26. The apparatus of claim 25, wherein the scan head further comprises an array of transducers to convert the radio frequency (RF) signal to an ultrasound signal and to transmit the ultrasound signal to the patient.

27. The apparatus of claim 26, wherein the transmitter is a low-voltage pulser.

28. The apparatus of claim 26, wherein the pulser is a high-voltage pulser, wherein the scan head further comprises a switch to isolate a transmit channel in the scan head from a receive channel in the scan head, and wherein the scan head further comprises a high-voltage multiplexer to select a set of transducers from among the array of transducers.

29. The apparatus of claim 26, wherein the array of transducers is further to receive the reflected ultrasound signal from the patient and to convert the reflected ultrasound signal to a second radio frequency (RF) signal.

30. The apparatus of claim 29, wherein the scan head further comprises receiver circuitry to amplify the second radio frequency (RF) signal.

31. The apparatus of claim 26, further comprising a programmable processor having data stored therein to cause the scan head to:
detect scan lines arising from an improper contact with the patient of the array of transducers; and
adjust the transmit power of the scan head based on the status of the transducer contact.

32. An article of manufacture, comprising:
a machine-accessible medium having data that, when accessed, results in a machine performing operations comprising:
selecting a first power mode for an ultrasound machine;
selecting an initial threshold value for a time for a battery in the home ultrasound machine;
determining an amount of power consumption for the battery;
based on the amount of power consumption, estimating an amount of energy remaining for the battery;
based on the amount of power consumption and the amount of energy remaining, estimating a time remaining for the battery;
if the amount of time remaining is greater than the initial threshold value, then maintaining operation of the ultrasound machine in the first power mode; and
if the amount of time remaining is less than or equal to the initial threshold value, then selecting a second power mode for the ultrasound machine.

33. The article of manufacture of claim 32, wherein the machine-accessible medium further includes data that, when accessed, results in a machine performing operations comprising reducing an intensity of a display for the ultrasound machine.

34. The article of manufacture of claim 32, wherein the machine-accessible medium further includes data that, when accessed, results in a machine performing operations comprising degrading an image quality to increase battery life for the ultrasound machine.

35. The article of manufacture of claim 32, wherein the machine-accessible medium further includes data that, when accessed, results in a machine performing operations comprising:
detecting scan lines arising from an improper contact with the patient of the array of transducers; and
selecting the second power mode for the ultrasound machine based on the improper contact.

36. A system for performing ultrasound examination of a patient, comprising:
an ultrasound machine;
a diagnosis station located remote from the ultrasound machine; and
a communication network coupled between the ultrasound machine and the diagnosis station,
wherein the diagnosis station is to transfer information associated with a scanning protocol for the ultrasound examination to the ultrasound machine via the communication network, and
wherein the ultrasound machine is to transfer measurement values acquired during the ultrasound examination to the diagnosis station via the communication network.

37. The system of claim 36, wherein the diagnosis station is to update lookup tables in the ultrasound machine via the communication network.

38. The system of claim 36, wherein the diagnosis station is to reconfigure hardware in the ultrasound machine via the communication network, wherein the hardware is to perform a first stage of beamforming on an ultrasound signal being reflected off the patient.

39. The system of claim 36, wherein the diagnosis station is to re-program at least one programmable processor in the ultrasound machine via the communication network, wherein the programmable processor is to perform a second stage of beamforming on the ultrasound signal being reflected off the patient.

40. The system of claim 36, wherein the ultrasound machine is located in a patient's home, a clinic, a vehicle, a physician's office, or a hospital.

41. The system of claim 40, wherein the ultrasound machine is located in a hospital emergency room.

42. An apparatus for generating multiple scan lines while performing ultrasound examination of a patient, the apparatus comprising:
a scan head to transmit an ultrasound signal and to receive a reflected ultrasound signal;
a pre-beamformer time delay lookup table (LUT) having stored therein K sets of different time delays; and
reconfigurable logic having K pre-beamformer processing units, each pre-beamformer processing unit to apply the K sets of different time delays to complex baseband signals produced from the reflected ultrasound signal to construct K scan lines.

43. The apparatus of claim 42, further comprising a buffer to drive the K pre-beamformer processing units.

44. The apparatus of claim 42, wherein the pre-beamformer time delay lookup table (LUT) is organized as R rows and N columns and wherein N and R represent a number of receive channels and a number of axial points corresponding to a penetration depth for the ultrasound signal transmitted from the scan head, respectively.

45. The apparatus of claim 42, wherein the pre-beamformer time delay lookup table (LUT) includes:
a control word lookup table having stored therein R reduced control words that are log₂ C-bit long; and
a codebook having stored therein log₂ C K-bit long codes, wherein the codebook is to decode at least one reduced control word to produce an original K-bit control word.

46. The apparatus of claim 45, further comprising variable length coding/run length coding (VLC/RLC) decoder coupled between the control word lookup table and the code book to decode at least one reduced control word prior to the codebook decoding at least one reduced control word to produce the original K-bit control word.

47. An apparatus for generating multiple scan lines while performing ultrasound examination of a patient, the apparatus comprising:
a scan head to transmit an ultrasound signal and to receive a reflected ultrasound signal;
a lookup table (LUT) having stored therein a time delay;
a memory having stored therein complex baseband signals produced from the reflected ultrasound signal; and
reconfigurable logic having a pre-beamformer processing unit to apply the time delay to complex baseband signals multiple times to construct multiple scan lines, respectively.
